(19) United States
(12) Patent Application Publication
(10) Pub. No.: US 2024/0270701 A1
(43) Pub. Date: Aug. 15, 2024

(54) BETA ADRENERGIC AGONIST AND METHODS OF USING THE SAME

(71) Applicant: Curasen Therapeutics, Inc., San Carlos, CA (US)

(72) Inventors: Jiaxin Yu, San Carlos, CA (US); Anthony P. Ford, San Carlos, CA (US); Wei Chen, San Carlos, CA (US)

(21) Appl. No.: 18/694,362

(22) PCT Filed: Sep. 22, 2022
(86) PCT No.: PCT/US2022/044437

§ 371 Date: Mar. 21, 2024

Related U.S. Application Data
(60) Provisional application No. 63/247,727, filed on Sep. 23, 2021.

(51) Int. Cl. C07D 241/42; A61K 31/4045; A61K 31/416; A61K 31/417; A61K 31/426; A61K 31/437; A61K 31/44; A61K 31/47; A61K 31/498; A61K 31/517; C07D 209/34; C07D 213/38; C07D 213/61; C07D 213/73; C07D 215/14; C07D 215/233; C07D 231/56; C07D 235/08; C07D 235/26; C07D 237/20; C07D 239/42; C07D 239/74; C07D 277/40; C07D 277/68; C07D 471/04

(52) U.S. Cl. CPC C07D 241/42; A61K 31/4045; A61K 31/416; A61K 31/417; A61K 31/426; A61K 31/437; A61K 31/44; A61K 31/47; A61K 31/498; A61K 31/517; C07D 209/34; C07D 213/38; C07D 213/61; C07D 213/73; C07D 215/14; C07D 215/233; C07D 231/56; C07D 235/08; C07D 235/26; C07D 237/20; C07D 239/42; C07D 239/74; C07D 277/40; C07D 277/68; C07D 471/04

(57) ABSTRACT

The present disclosure is directed to chemical compounds and to the use of such compounds in the treatment of diseases associated with an adrenergic receptor. An advantage of the compounds provided herein is that a broad range of pharmacological activities is possible, consistent with modulation of beta-adrenergic receptors. In addition, the disclosure provides methods of using the compounds described herein for the treatment of diseases associated with an adrenergic receptor, including but not limited to neurodegenerative diseases and disorders.

### CROSS-REFERENCE TO RELATED APPLICATIONS

This application claims the benefit of priority under 35 U.S.C. § 119(e) of U.S. Provisional Application No. 63/247,727, filed Sep. 23, 2021. The disclosure of the prior application is considered part of and is herein incorporated by reference in the disclosure of this application in its entirety.

### FIELD

The present disclosure relates generally to chemical compounds and, in some embodiments, to beta-adrenergic agonists and uses in the treatment of diseases associated with an adrenergic receptor.

### BACKGROUND

PCT Application Publication Number WO 2017/197324 discloses “[a]drenergic receptor modulating compounds and methods . . . of treating a subject for a disease or condition associated with an adrenergic receptor including administering a therapeutically effective amount of the subject compound.”

United States Patent Application Publication Number 2013/0096126 discloses “a method for enhancing learning or memory of both in a mammal having impaired learning or memory or both from a neuro-degenerative disorder, which entails the step of administering at least one compound or a salt thereof which is a β1-adrenergic receptor agonist, partial agonist or receptor ligand in an amount effective to improve the learning or memory or both of said mammal.”

United States Patent Application Publication Number 2014/0235726 discloses “a method of improving cognition in a patient with Down syndrome, which entails administering one or more 2 adrenergic receptor agonists to the patient in an amount and with a frequency effective to improve cognition of the patient as measured by contextual learning tests.”

United States Patent Application Publication Number 2016/0184241 discloses “a method of improving cognition in a patient with Down syndrome, which entails intranasally administering one or more β2-ADR agonists or pharmaceutically-acceptable salts of either or both to the patient in an amount and with a frequency effective to improve cognition of the patient as measured contextual learning tests.”

### SUMMARY

The present disclosure relates to small molecule compounds that modulate adrenergic receptors, methods of preparation of the compounds, pharmaceutical compositions comprising the compounds, and their use in medical therapy. In particular, the present disclosure provides compounds which find utility as beta-adrenergic agonists. An advantage of the compounds provided herein is that a broad range of pharmacological activities is possible, consistent with modulation of beta-adrenergic receptors. In addition, the disclosure provides methods of using the compounds described herein for the treatment of diseases associated with an adrenergic receptor.

In has now been found that compounds of the present disclosure, and pharmaceutically acceptable compositions thereof, are effective as beta-adrenergic agonists. Such compounds of the disclosure have the general formula:

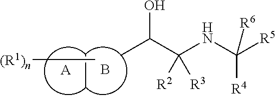

or a pharmaceutically acceptable salt thereof, wherein each variable is as defined and described herein.

Compounds of the present disclosure, and pharmaceutically acceptable compositions thereof, are useful for treating a variety of diseases, disorders, or conditions, associated with aberrant beta-adrenergic receptor activity. Such diseases, disorders, or conditions include those described herein.

Compounds provided by the present disclosure are also useful for the study of beta-adrenergic receptors in biological and pathological phenomena and the comparative evaluation of new beta-adrenergic agonists or other regulators of beta-adrenergic receptors, signaling pathways, and neurotransmitter levels in vitro or in vivo.

### DETAILED DESCRIPTION

In the following disclosure, certain specific details are set forth in order to provide a thorough understanding of various embodiments. However, one skilled in the art will understand that the methods and uses described herein may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the disclosures herein.

Reference throughout this specification to “an embodiment” or “some embodiments” means that a particular feature, structure, or characteristic described in connection with the embodiment is included in at least one embodiment. Thus, the appearances of the phrase “in some embodiments” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

### Definitions

Compounds of the present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1 to 6 aliphatic carbon atoms. In some embodiments, aliphatic groups contain 1 to 5 aliphatic carbon atoms. In other embodiments, aliphatic groups contain 1 to 4 aliphatic carbon atoms. In still other embodiments, aliphatic groups contain 1 to 3 aliphatic carbon atoms, and in yet other embodiments, aliphatic groups contain 1 to 2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7 to 12 ring members and 0 to 4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

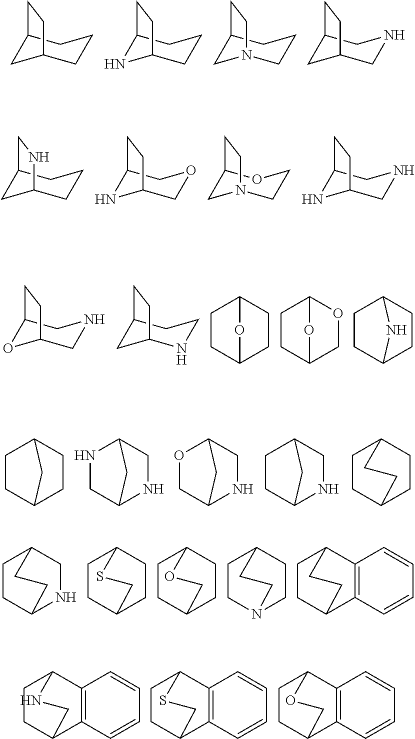

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen; or an oxygen, sulfur, nitrogen, phosphorus, or silicon atom in a heterocyclic ring.

The term “unsaturated,” as used herein, means that a moiety has one or more units of unsaturation.

As used herein, the term “bivalent Cis (or C₁-6) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

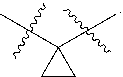 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of 4 to 14 ring members, wherein at least one ring in the system is aromatic and wherein each ring in the system contains 3 to 7 ring members. The term “aryl” may be used interchangeably with the term “aryl ring.” In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from 1 to 5 heteroatoms. The term “heteroatom” in the context of “heteroaryl” particularly includes, but is not limited to, nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic or bicyclic. A heteroaryl ring may include one or more oxo (═O) or thioxo (═S) substituent. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7- to 10-membered bicyclic heterocyclic moiety that is either saturated or partially unsaturated, and having, in addition to carbon atoms, one or more, preferably 1 to 4, heteroatoms, as defined above. When used in reference to a ring atom of a heterocycle, the term “nitrogen” includes a substituted nitrogen. As an example, in a saturated or partially unsaturated ring may have 0 to 3 heteroatoms selected from oxygen, sulfur or nitrogen.

A heterocyclic ring can be attached to a provided compound at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic, bridged bicyclic, or spirocyclic. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the disclosure may contain “substituted” moieties. In general, the term “substituted” means that one or more hydrogens of the designated moiety are replaced with a suitable substituent. Unless otherwise indicated, an “optionally substituted” group may have a suitable substituent at one or more substitutable position of the group, and when more than one position in any given structure is substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₆R^{o}; —(CH₂)₀₋₆OR^{o}; —O(CH₂)₀₋₆R^{o}; —O—(CH₂)₀₋₆C(O)OR^{o}; —(CH₂)₀₋₆CH(OR^{o})₂; —(CH₂)₀₋₆SR^{o}; —(CH₂)₀₋₆Ph, which Ph may be substituted with R^{o}; —(CH₂)₀₋₆O(CH₂)₀₋₁Ph which Ph may be substituted with R^{o}; —CH═CHPh, which Ph may be substituted with R^{o}; —(CH₂)₀₋₆O(CH₂)₀₋₁-pyridyl which pyridyl may be substituted with R^{o}; —NO₂; —CN; —N₃; —(CH₂)₀₋₆N(R^{o})₂; —(CH₂)₀₋₆N(R^{o})C(O)R^{o}; —N(R^{o})C(S)R^{o}; —(CH₂)₀₋₆N(R^{o})C(O)NR^{o}₂; —N(R^{o})C(S)NR^{o}₂; —(CH₂)₀₋₆N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₆C(O)R^{o}; —C(S)R^{o}; —(CH₂)₀₋₆C(O)OR^{o}; —(CH₂)₀₋₆C(O)SR^{o}; —(CH₂)₀₋₆C(O)OSiR^{o}₃; —(CH₂)₀₋₆OC(O)R^{o}; —OC(O)(CH₂)₀₋₆SR^{o}, —(CH₂)₀₋₆SC(O)R^{o}; —(CH₂)₀₋₆C(O)NR^{o}₂; —C(S)NR^{o}₂; —C(S)SR^{o}; —SC(S)SR^{o}, —(CH₂)₀₋₆OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₆SSR^{o}; —(CH₂)₀₋₆S(O)₂R^{o}; —(CH₂)₀₋₆S(O)₂OR^{o}; —(CH₂)₀₋₆OS(O)₂R^{o}; —S(O)₂NR^{o}₂; —(CH₂)₀₋₆S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —P(O)(OR^{o})₂; —OP(O)(R^{o})OR^{o}; —OP(O)R^{o}₂; —OP(O)(OR^{o})₂; SiR^{o}₃; —(C₁₋₄ straight or branched alkylene)O—N(R^{o})₂; or —(C₁₋₄ straight or branched alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5- to 6-membered heteroaryl ring), or a 5- to 6-membered saturated, partially unsaturated, or aryl ring having 0 to 4 heteroatoms independently selected from nitrogen, oxygen, or sulfur, or, notwithstanding the definition above, two independent occurrences of R^{o}, taken together with their intervening atom(s), form a 3- to 12-membered saturated, partially unsaturated, or aryl mono- or bicyclic ring having 0 to 4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂, —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —N₀₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5- to 6-membered saturated, partially unsaturated, or aryl ring having 0 to 4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5- to 6-membered saturated, partially unsaturated, or aryl ring having 0 to 4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5- to 6-membered saturated, partially unsaturated, or aryl ring having 0 to 4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0 to 4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5- to 6-membered saturated, partially unsaturated, or aryl ring having 0 to 4 heteroatoms independently selected from nitrogen, oxygen, or sulfur, or, notwithstanding the definition above, two independent occurrences of R^{†}, taken together with their intervening atom(s) form an unsubstituted 3- to 12-membered saturated, partially unsaturated, or aryl monocyclic or bicyclic ring having 0 to 4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0 to 4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, which is incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, “prodrug” refers to a derivative of an active agent that requires a transformation within the body to release the active agent. In certain embodiments, the transformation is an enzymatic transformation. Prodrugs are frequently, although not necessarily, pharmacologically inactive until converted to the active agent. “Promoiety” refers to a form of protecting group that, when used to mask a functional group within an active agent, converts the active agent into a prodrug. In some cases, the promoiety will be attached to the drug via bond(s) that are cleaved by enzymatic or non-enzymatic means in vivo. Any convenient prodrug forms of the subject compounds can be prepared, e.g., according to the strategies and methods described by Rautio et al. (“Prodrugs: design and clinical applications”, Nature Reviews Drug Discovery 7, 255-270 (February 2008)).

As used herein, the term “provided compound” refers to any genus, subgenus, and/or species set forth herein.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of the disclosure are within the scope of the disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure.

### Compounds of the Present Disclosure

Compounds of the present disclosure, and compositions thereof, are useful as beta adrenergic modulators. In some embodiments, a provided compound modulates a beta-adrenergic receptor.

In certain embodiments a compound as disclosed herein is an agonist, partial agonist or antagonist of an adrenergic receptor. In some embodiments the compound is a β1-adrenergic receptor agonist, β2-adrenertic receptor agonist, or non-selective β1/β2-adrenergic receptor agonist. In some embodiments the compound is a β1-adrenergic receptor agonist. In some embodiments the compound is a β2-adrenergic receptor agonist. In some embodiments the compound is a non-selective β1/β2-adrenergic agonist.

As described herein, a structure depicted as

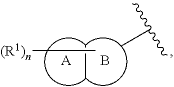

includes for example, structures

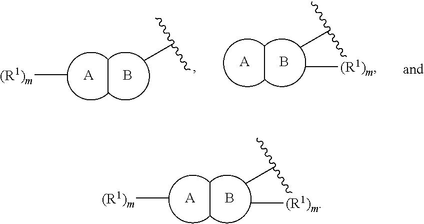

The present disclosure provides a compound of formula I-a:

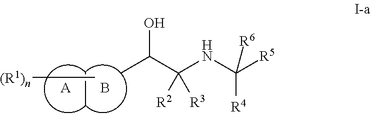

or a pharmaceutically acceptable salt thereof, wherein:
each R¹ is independently hydrogen, halogen, R^{A}, —CN, —NO₂, —SF₅, —OR, —N(R)₂, —SO₂R, —C(O)R, —C(O)N(R)₂, —NRC(O)R, —NRCO₂R, or —CO₂R;
each R is independently hydrogen or an optionally substituted group selected from C₁-6 aliphatic, phenyl, a 3-8 membered saturated or partially unsaturated carbocyclic or heterocyclic ring having 1-3 heteroatoms independently selected from nitrogen, oxygen, or sulfur, a 5-6 membered heteroaryl ring having 1-3 heteroatoms independently selected from nitrogen, oxygen, and sulfur, or:
   two R groups on the same nitrogen atom are optionally taken together with their intervening atoms to form an optionally substituted 3-7 membered saturated or partially unsaturated heterocyclic ring having 0-2 heteroatoms, in addition to the nitrogen atom from which the two R groups are attached, independently selected from nitrogen, oxygen, and sulfur;
each R^{A} is independently an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R^{A} groups on the same carbon atom are optionally taken together with their intervening atoms to form an optionally substituted 3-6 membered saturated or partially unsaturated carbocyclic or heterocyclic ring having 1-3 heteroatoms independently selected from nitrogen, oxygen, and sulfur;
R² and R³ are each independently hydrogen or optionally substituted C₁₋₆ aliphatic, or:
   R² and R³ are optionally taken together with the carbon atom they are attached to form an optionally substituted 3-6 membered saturated or partially unsaturated carbocyclic or heterocyclic ring having 1-2 heteroatoms independently selected from nitrogen, oxygen, and sulfur;
R⁴ is an optionally substituted C₁₋₆ aliphatic;
R⁵ is hydrogen or an optionally substituted C₁₋₆ aliphatic;
R⁶ is an optionally substituted group selected from C₂₋₉ aliphatic, phenylC₀₋₃alkyl, heterocyclylC₀₋₃alkyl having 1-2 heteroatoms independently selected from nitrogen, oxygen, and sulfur, and heteroarC₀₋₃alkyl having 1-2 heteroatoms independently selected from nitrogen, oxygen, and sulfur;
Ring A and Ring B, independently, are fused rings selected from benzo, 5-6 membered heteroaryl containing 1-4 heteroatoms independently selected from nitrogen, oxygen, and sulfur, and 5-7 membered saturated or partially unsaturated carbocyclyl or heterocyclyl having 1-3 heteroatoms independently selected from nitrogen, oxygen, and sulfur; and
n is 0, 1, 2, 3, 4, 5, or 6.

In some embodiments, the present disclosure provides a compound of formula I-a, wherein Ring A is pyrazolylenyl as shown, to provide a compound of formula I-a-1:

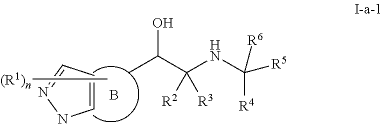

or a pharmaceutically acceptable salt thereof, wherein each of Ring B, R¹, R², R³, R⁴, R⁵, R⁶, and n is as defined above and described in embodiments herein, both singly and in combination.

In some embodiments, the present disclosure provides a compound of formula I-a, wherein R² and R³ are hydrogen as shown, to provide a compound of formula I-a-2:

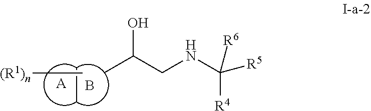

or a pharmaceutically acceptable salt thereof, wherein each of Ring A, Ring B, R¹, R⁴, R⁵, R⁶, and n is as defined above and described in embodiments herein, both singly and in combination.

In some embodiments, the present disclosure provides a compound of formula I-a, wherein Ring A is pyrazolylenyl, and R² and R³ are hydrogen as shown, to provide a compound of formula I-a-3:

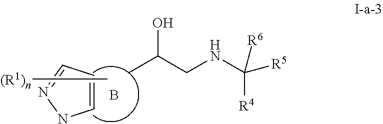

or a pharmaceutically acceptable salt thereof, wherein each of Ring B, R¹, R⁴, R⁵, R⁶, and n is as defined above and described in embodiments herein, both singly and in combination.

In some embodiments, the present disclosure provides a compound of formula I-a, wherein Ring B is benzo as shown below, to provide a compound of formula I-a-4:

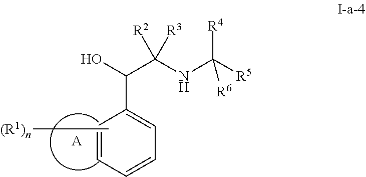

or a pharmaceutically acceptable salt thereof, wherein each of Ring A, R¹, R², R³, R⁴, R⁵, R⁶, and n is as defined above and described in embodiments herein, both singly and in combination.

In some embodiments, the present disclosure provides a compound of formula I-a, wherein Ring B is benzo and R² and R³ are hydrogen, as shown below, to provide a compound of formula I-a-5:

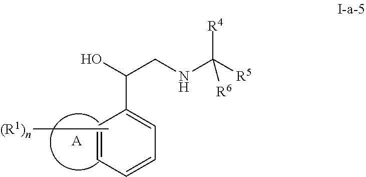

or a pharmaceutically acceptable salt thereof, wherein each of Ring A, R¹, R⁴, R⁵, R⁶, and n is as defined above and described in embodiments herein, both singly and in combination.

In some embodiments, the present disclosure provides a compound of formula I-a, wherein Ring A is pyrazolylenyl as shown below, to provide a compound of formula I-a-6:

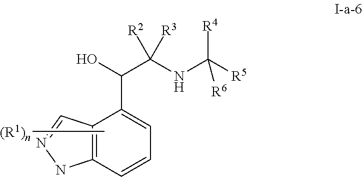

or a pharmaceutically acceptable salt thereof, wherein each of R¹, R², R³, R⁴, R⁵, R⁶, and n is as defined above and described in embodiments herein, both singly and in combination.

In some embodiments, the present disclosure provides a compound of formula I-a, wherein Ring A is pyrazolylenyl as shown below, to provide a compound of formula I-a-7:

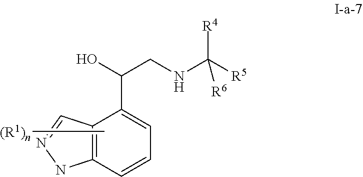

or a pharmaceutically acceptable salt thereof, wherein each of R¹, R⁴, R⁵, R⁶, and n is as defined above and described in embodiments herein, both singly and in combination.

The present disclosure provides a compound of formula I-b:

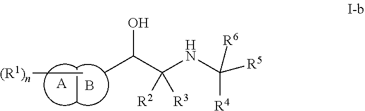

or a pharmaceutically acceptable salt thereof, wherein:
each R¹ is independently hydrogen, halogen, R^{A}, —CN, —NO₂, —SF₅, —OR, —N(R)₂, —SO₂R, —C(O)R, —C(O)N(R)₂, —NRC(O)R, —NRCO₂R, or —CO₂R;
each R is independently hydrogen or an optionally substituted group selected from C₁₋₆ aliphatic, phenyl, a 3-8 membered saturated or partially unsaturated carbocyclic or heterocyclic ring having 1-3 heteroatoms independently selected from nitrogen, oxygen, or sulfur, a 5-6 membered heteroaryl ring having 1-3 heteroatoms independently selected from nitrogen, oxygen, and sulfur, or:
   two R groups on the same nitrogen atom are optionally taken together with their intervening atoms to form an optionally substituted 3-7 membered saturated or partially unsaturated heterocyclic ring having 0-2 heteroatoms, in addition to the nitrogen atom from which the two R groups are attached, independently selected from nitrogen, oxygen, and sulfur;
each R^{A} is independently an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R^{A} groups on the same carbon atom are optionally taken together with their intervening atoms to form an optionally substituted 3-6 membered saturated or partially unsaturated carbocyclic or heterocyclic ring having 1-3 heteroatoms independently selected from nitrogen, oxygen, and sulfur;
R² and R³ are each independently hydrogen or optionally substituted C₁₋₆ aliphatic, or:
   R² and R³ are optionally taken together with the carbon atom they are attached to form an optionally substituted 3-6 membered saturated or partially unsaturated carbocyclic or heterocyclic ring having 1-2 heteroatoms independently selected from nitrogen, oxygen, and sulfur;
R⁴ and R⁵ are each independently hydrogen or optionally substituted C₁₋₆ aliphatic, or:
   R⁴ and R⁵ are optionally taken together with the carbon atom they are attached to form an optionally substituted 3-6 membered saturated or partially unsaturated carbocyclic or heterocyclic ring having 1-2 heteroatoms independently selected from nitrogen, oxygen, and sulfur;
R⁶ is an optionally substituted group selected from C₁₋₉ aliphatic, phenylC₀₋₃alkyl, heterocyclylC₀₋₃alkyl having 1-2 heteroatoms independently selected from nitrogen, oxygen, and sulfur, and heteroarC₀₋₃alkyl having 1-2 heteroatoms independently selected from nitrogen, oxygen, and sulfur;
Ring A and Ring B, independently, are fused rings selected from benzo, 5-6 membered heteroaryl containing 1-4 heteroatoms independently selected from nitrogen, oxygen, and sulfur, and 5-7 membered saturated or partially unsaturated carbocyclyl or heterocyclyl having 1-3 heteroatoms independently selected from nitrogen, oxygen, and sulfur; and
n is 2, 3, 4, 5, or 6,
wherein when Ring A is a fused 5-membered heterocyclic ring, it is not a 5-membered heterocyclic ring containing one nitrogen.

In some embodiments, the present disclosure provides a compound of formula I-b, wherein Ring A is pyrazolylenyl as shown below, to provide a compound of formula I-b-1:

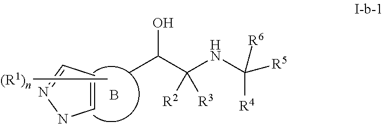

or a pharmaceutically acceptable salt thereof, wherein each of Ring B, R¹, R², R³, R⁴, R⁵, R⁶, and n is as defined above and described in embodiments herein, both singly and in combination.

In some embodiments, the present disclosure provides a compound of formula I-b, wherein R² and R³ are hydrogen as shown, to provide a compound of formula I-b-2:

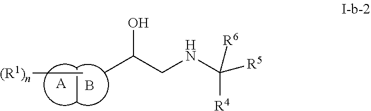

or a pharmaceutically acceptable salt thereof, wherein each of Ring A, Ring B, R¹, R⁴, R⁵, R⁶, and n is as defined above and described in embodiments herein, both singly and in combination.

In some embodiments, the present disclosure provides a compound of formula I-b, wherein Ring A is pyrazolylenyl and R² and R³ are hydrogen, as shown below, to provide a compound of formula I-b-3:

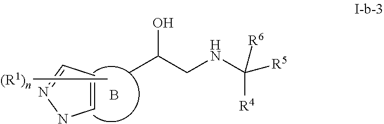

or a pharmaceutically acceptable salt thereof, wherein each of Ring B, R¹, R⁴, R⁵, R⁶, and n is as defined above and described in embodiments herein, both singly and in combination.

In some embodiments, the present disclosure provides a compound of formula I-b, wherein Ring B is benzo as shown below, to provide a compound of formula I-b-4:

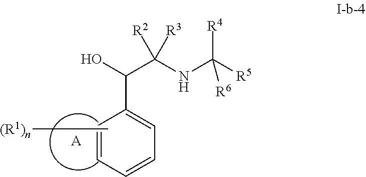

or a pharmaceutically acceptable salt thereof, wherein each of Ring A, R¹, R², R³, R⁴, R⁵, R⁶, and n is as defined above and described in embodiments herein, both singly and in combination.

In some embodiments, the present disclosure provides a compound of formula I-b, wherein Ring B is benzo and R² and R³ are hydrogen, as shown below, to provide a compound of formula I-b-5:

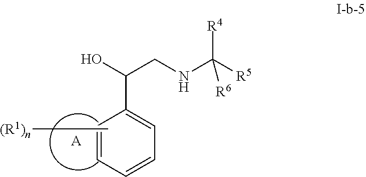

or a pharmaceutically acceptable salt thereof, wherein each of Ring A, R¹, R⁴, R⁵, R⁶, and n is as defined above and described in embodiments herein, both singly and in combination.

In some embodiments, the present disclosure provides a compound of formula I-b, wherein Ring A is pyrazolylenyl as shown below, to provide a compound of formula I-b-6:

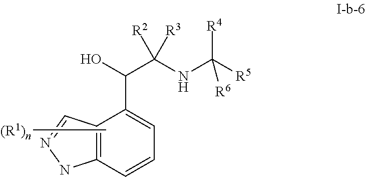

or a pharmaceutically acceptable salt thereof, wherein each of R¹, R², R³, R⁴, R⁵, R⁶, and n is as defined above and described in embodiments herein, both singly and in combination.

In some embodiments, the present disclosure provides a compound of formula I-b, wherein Ring A is pyrazolylenyl as shown below, to provide a compound of formula I-b-7:

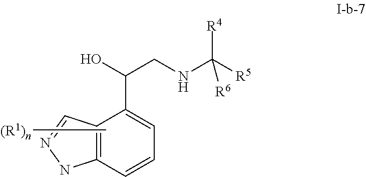

or a pharmaceutically acceptable salt thereof, wherein each of R¹, R⁴, R⁵, R⁶, and n is as defined above and described in embodiments herein, both singly and in combination.

As defined above and described herein, each R¹ is independently hydrogen, halogen, R^{A}, —CN, —NO₂, —SF₅, —OR, —N(R)₂, —SO₂R, —C(O)R, —C(O)N(R)₂, —NRC(O)R, —NRCO₂R, or —CO₂R.

In some embodiments, R¹ is hydrogen. In some embodiments, R¹ is halogen. In some embodiments, R¹ is R^{A}. In some embodiments, R¹ is —CN. In some embodiments, R¹ is —NO₂. In some embodiments, R¹ is —SF₅. In some embodiments, R¹ is —OR. In some embodiments, R¹ is —N(R)₂. In some embodiments, R¹ is —SO₂R. In some embodiments, R¹ is —C(O)R. In some embodiments, R¹ is —C(O)N(R)₂. In some embodiments, R¹ is —NRC(O)R. In some embodiments, R¹ is —NRCO₂R. In some embodiments, R¹ is —CO₂R. In some embodiments, R¹ is chloro. In some embodiments, R¹ is fluoro. In some embodiments, R¹ is methyl. In some embodiments, R¹ is methoxy. In some embodiments, R¹ is —OCHF₂.

In some embodiments, R¹ is selected from those depicted in Tables 1A-C, below.

As defined above and described herein, each R is independently hydrogen or an optionally substituted group selected from C₁₋₆ aliphatic, phenyl, a 3-8 membered saturated or partially unsaturated carbocyclic or heterocyclic ring having 1-3 heteroatoms independently selected from nitrogen, oxygen, or sulfur, a 5-6 membered heteroaryl ring having 1-3 heteroatoms independently selected from nitrogen, oxygen, and sulfur, or two R groups on the same nitrogen atom are optionally taken together with their intervening atoms to form an optionally substituted 3-7 membered saturated or partially unsaturated heterocyclic ring having 0-2 heteroatoms, in addition to the nitrogen atom from which the two R groups are attached, independently selected from nitrogen, oxygen, and sulfur.

In some embodiments, R is hydrogen. In some embodiments, R is an optionally substituted C₁₋₆ aliphatic (e.g., methyl, ethyl, isopropyl, cyclopropyl, cyclobutyl, trifluoromethyl, etc.). In some embodiments, R is an optionally substituted phenyl. In some embodiments, R is an optionally substituted 3-8 membered saturated or partially unsaturated carbocyclic ring. In some embodiments, R is an optionally substituted 3-8 membered saturated or partially unsaturated heterocyclic ring having 1-3 heteroatoms independently selected from nitrogen, oxygen, or sulfur. In some embodiments, R is an optionally substituted 5-6 membered heteroaryl ring having 1-3 heteroatoms independently selected from nitrogen, oxygen. In some embodiments, two R groups on the same nitrogen atom are optionally taken together with their intervening atoms to form an optionally substituted 3-7 membered saturated or partially unsaturated heterocyclic ring having 0-2 heteroatoms, in addition to the nitrogen atom from which the two R groups are attached, independently selected from nitrogen, oxygen, and sulfur.

In some embodiments, R¹ is selected from those depicted in Tables 1A-C, below.

As defined above and described herein, each R^{A} is independently an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or two R^{A} groups on the same carbon atom are optionally taken together with their intervening atoms to form an optionally substituted 3-6 membered saturated or partially unsaturated carbocyclic or heterocyclic ring having 1-3 heteroatoms independently selected from nitrogen, oxygen, and sulfur.

In some embodiments, R^{A} is an optionally substituted C₁₋₆ aliphatic (e.g., methyl, ethyl, isopropyl, cyclopropyl, cyclobutyl, trifluoromethyl, etc.). In some embodiments, R^{A} is an optionally substituted phenyl. In some embodiments, R^{A} is an optionally substituted 4-7 membered saturated or partially unsaturated carbocyclic ring. In some embodiments, R^{A} is an optionally substituted 4-7 membered saturated or partially unsaturated heterocyclic ring having 1-2 heteroatoms independently selected from nitrogen, oxygen, and sulfur. In some embodiments, R^{A} is an optionally substituted 5-6 membered heteroaryl ring having 1-4 heteroatoms independently selected from nitrogen, oxygen, and sulfur. In some embodiments, two R^{A} groups on the same carbon atom are optionally taken together with their intervening atoms to form an optionally substituted 3-6 membered saturated or partially unsaturated carbocyclic ring. In some embodiments, two R^{A} groups on the same carbon atom are optionally taken together with their intervening atoms to form an optionally substituted 3-6 membered saturated or partially unsaturated heterocyclic ring having 1-3 heteroatoms independently selected from nitrogen, oxygen, and sulfur.

In some embodiments, R^{A} is selected from those depicted in Tables 1A-C, below.

As defined above and described herein, R² and R³ are each independently hydrogen or optionally substituted C₁₋₆ aliphatic, or R² and R³ are optionally taken together with the carbon atom to which they are attached to form an optionally substituted 3-6 membered saturated or partially unsaturated carbocyclic or heterocyclic ring having 1-2 heteroatoms independently selected from nitrogen, oxygen, and sulfur.

In some embodiments, R² is hydrogen. In some embodiments, R² is an optionally substituted C₁₋₆ aliphatic (e.g., methyl, ethyl, isopropyl, cyclopropyl, cyclobutyl, trifluoromethyl, etc.).

In some embodiments, R³ is hydrogen. In some embodiments, R³ is an optionally substituted C₁₋₆ aliphatic (e.g., methyl, ethyl, isopropyl, cyclopropyl, cyclobutyl, trifluoromethyl, etc.).

In some embodiments, R² and R³ are optionally taken together with the carbon atom to which they are attached to form an optionally substituted 3-6 membered saturated or partially unsaturated carbocyclic ring (e.g., cyclopropylenyl, cyclobutylenyl, etc.). In some embodiments, R² and R³ are optionally taken together with the carbon atom to which they are attached to form an optionally substituted 3-6 membered saturated or partially unsaturated heterocyclic ring having 1-2 heteroatoms independently selected from nitrogen, oxygen, and sulfur (e.g., tetrahydrofuranylenyl, tetrahydro-2H-pyranylenyl, pyrrolidinylenyl, piperidinylenyl, etc.).

In some embodiments, R² and R³ are selected from those depicted in Tables 1A-C, below.

As defined above and described herein, R⁴ is hydrogen or an optionally substituted C₁₋₆ aliphatic.

In some embodiments, R⁴ is hydrogen. In some embodiments, R⁴ is an optionally substituted C₁₋₆ aliphatic (e.g., methyl, ethyl, isopropyl, cyclopropyl, cyclobutyl, trifluoromethyl, etc.). In some embodiments, R⁴ is methyl. In some embodiments, R⁴ is ethyl. In some embodiments, R⁴ is isopropyl. In some embodiments, R⁴ is cyclopropyl. In some embodiments, R⁴ is tert-butyl.

As defined above and described herein, R⁵ is hydrogen or an optionally substituted C₁₋₆ aliphatic.

In some embodiments, R⁵ is hydrogen. In some embodiments, R⁵ is an optionally substituted C₁₋₆ aliphatic (e.g., methyl, ethyl, isopropyl, cyclopropyl, cyclobutyl, trifluoromethyl, etc.). In some embodiments, R⁵ is methyl. In some embodiments, R⁵ is ethyl. In some embodiments, R⁵ is isopropyl. In some embodiments, R⁵ is cyclopropyl. In some embodiments, R⁵ is tert-butyl.

As defined above and described herein, R⁴ and R⁵ are optionally taken together with the carbon atom to which they are attached to form an optionally substituted 3-6 membered saturated or partially unsaturated carbocyclic or heterocyclic ring having 1-2 heteroatoms independently selected from nitrogen, oxygen, and sulfur.

In some embodiments, R⁴ and R⁵ are optionally taken together with the carbon atom to which they are attached to form an optionally substituted 3-6 membered saturated or partially unsaturated carbocyclic or heterocyclic ring having 1-2 heteroatoms independently selected from nitrogen, oxygen, and sulfur.

In some embodiments, R⁴ and R⁵ are selected from those depicted in Tables 1A-C, below.

As defined above and described herein, R⁶ is an optionally substituted group selected from C₁₋₉ aliphatic, phenylC₀₋₃alkyl, heterocyclylC₀₋₃alkyl having 1-2 heteroatoms independently selected from nitrogen, oxygen, and sulfur, and heteroarC₀₋₃alkyl having 1-2 heteroatoms independently selected from nitrogen, oxygen, and sulfur.

In some embodiments, R⁶ is an optionally substituted C₁₋₉ aliphatic (e.g., methyl, ethyl, isopropyl, cyclopropyl, cyclobutyl, trifluoromethyl, etc.). In some embodiments, R⁶ is an optionally substituted C₂₋₉ aliphatic (e.g., ethyl, isopropyl, cyclopropyl, cyclobutyl, trifluoromethyl, etc.). In some embodiments, R⁶ is an optionally substituted phenylC₀₋₃alkyl. In some embodiments, R⁶ is an optionally substituted heterocyclylC₀₋₃alkyl having 1-2 heteroatoms independently selected from nitrogen, oxygen, and sulfur. In some embodiments, R⁶ is an optionally substituted heteroarC₀₋₃alkyl having 1-2 heteroatoms independently selected from nitrogen, oxygen, and sulfur. In some embodiments, R⁶ is methyl. In some embodiments, R⁶ is ethyl. In some embodiments, R⁶ is isopropyl. In some embodiments, R⁶ is cyclopropyl. In some embodiments, R⁶ is tert-butyl. In some embodiments, R⁶ is an optionally substituted benzyl. In some embodiments, R⁶ is benzyl. In some embodiments, R⁶ is para-methoxybenzyl.

In some embodiments, R⁶ is selected from those depicted in Tables 1A-C, below.

As defined above and described herein, Ring A and Ring B, independently, are fused rings selected from benzo, 5-6 membered heteroaryl containing 1-4 heteroatoms independently selected from nitrogen, oxygen, and sulfur, and a 5 to 7-membered saturated or partially unsaturated carbocyclyl or heterocyclyl having 1-3 heteroatoms independently selected from nitrogen, oxygen, and sulfur.

In some embodiments, Ring A is benzo. In some embodiments, Ring A is a 5-6 membered heteroaryl containing 1-4 heteroatoms independently selected from nitrogen, oxygen, and sulfur. In some embodiments, Ring A is a 5 to 7-membered saturated or partially unsaturated carbocyclyl. In some embodiments, Ring A is a 5 to 7-membered saturated or partially unsaturated heterocyclyl having 1-3 heteroatoms independently selected from nitrogen, oxygen, and sulfur. In some embodiments, Ring A is pyrrolidinylenyl. In some embodiments, Ring A is pyrazolylenyl. In some embodiments, Ring A is pyridinylenyl. In some embodiments, Ring A is pyrimidinylenyl.

In some embodiments, Ring B is benzo. In some embodiments, Ring B is a 5-6 membered heteroaryl containing 1-4 heteroatoms independently selected from nitrogen, oxygen, and sulfur. In some embodiments, Ring B is a 5 to 7-membered saturated or partially unsaturated carbocyclyl. In some embodiments, Ring B is a 5 to 7-membered saturated or partially unsaturated heterocyclyl having 1-3 heteroatoms independently selected from nitrogen, oxygen, and sulfur. In some embodiments, Ring B is pyridinylenyl.

In some embodiments, when Ring A is a fused 5-membered heterocyclic ring, it is not a 5-membered heterocyclic ring containing one nitrogen.

In some embodiments, Ring A and Ring B are selected from those depicted in Tables 1A-C, below.

As defined above and described herein, n is 0, 1, 2, 3, 4, 5, or 6.

In some embodiments, n is 0. In some embodiments, n is 1. In some embodiments, n is 2. In some embodiments, n is 3. In some embodiments, n is 4. In some embodiments, n is 5. In some embodiments, n is 6.

Exemplary compounds of the present disclosure are set forth in the tables below.

TABLE 1A
Exemplary compounds

| | | MS |
|---|---|---|
| I-# | Structure | (M + H) |
| I-1 | 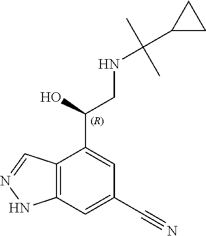 | 285.4 |
| I-2 | 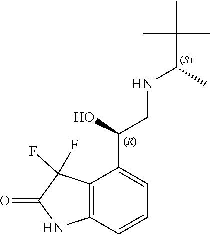 | 313.3 |
| I-3 | 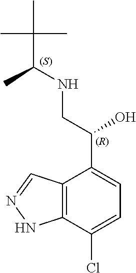 | 296.8 |
| I-4 | 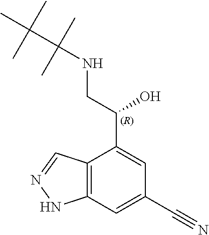 | 301.3 |
| I-5 | 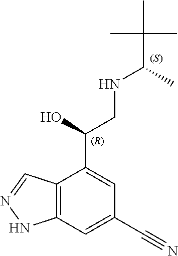 | 287.4 |
| I-6 | 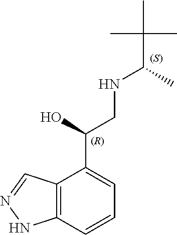 | 262.4 |
| I-7 | 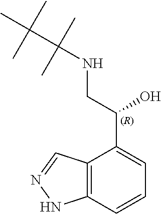 | 276.4 |
| I-8 | 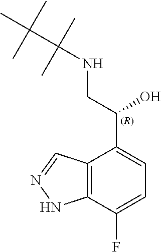 | 294.3 |
| I-9 | 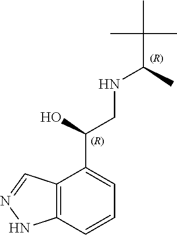 | 262.3 |
| I-10 | 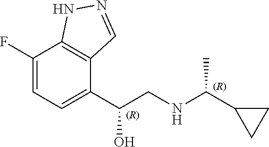 | 264.3 |
| I-11 | 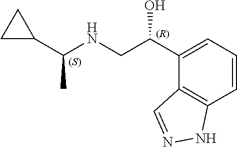 | 246.3 |
| I-12 | 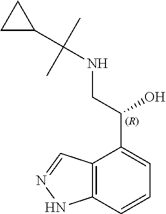 | 260.3 |
| I-13 | 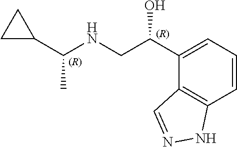 | 246.4 |
| I-14 | 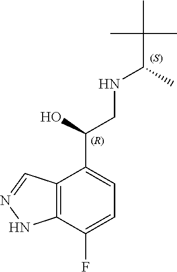 | 280.4 |
| I-15 | 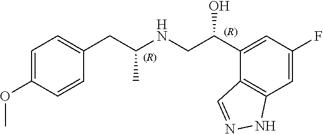 | 344.4 |
| I-16 | 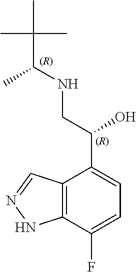 | 280.4 |
| I-17 | 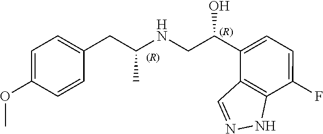 | 344.4 |
| I-18 | 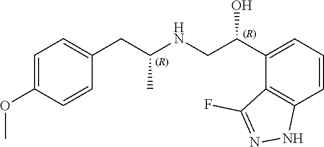 | 344.4 |
| I-19 | 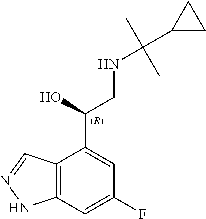 | 278.3 |
| I-20 | 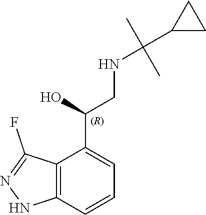 | 278.3 |
| I-21 | 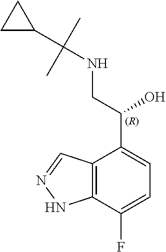 | 278.3 |
| I-22 | 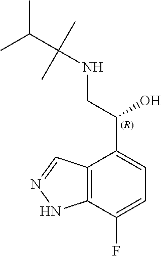 | 280.4 |
| I-23 | 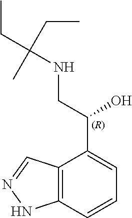 | 262.4 |
| I-24 | 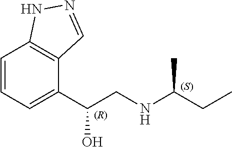 | 234.3 |
| I-25 | 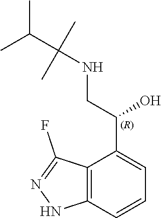 | 280.3 |
| I-26 | 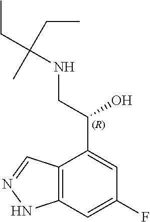 | 280.4 |
| I-27 | 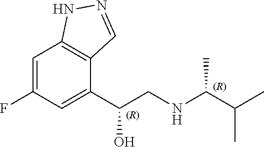 | 266.3 |
| I-28 | 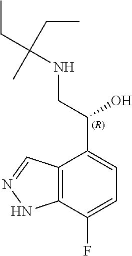 | 280.3 |
| I-29 | 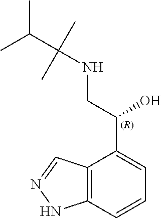 | 262.4 |
| I-30 | 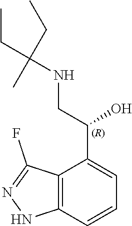 | 280.4 |
| I-31 | 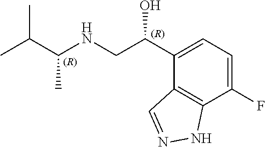 | 266.3 |
| I-32 | 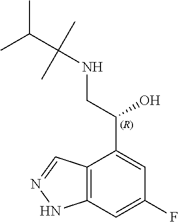 | 280.4 |
| I-33 | 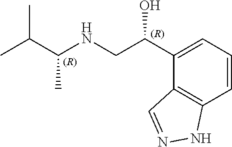 | 248.4 |
| I-34 | 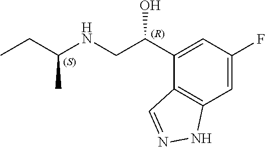 | 252.3 |
| I-35 | 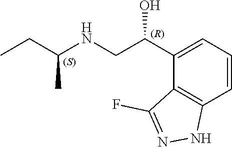 | 252.3 |
| I-36 | 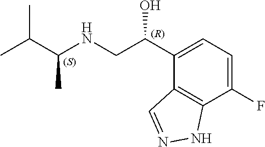 | 266.3 |
| I-37 | 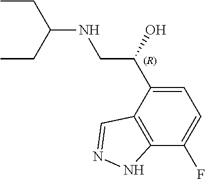 | 266.3 |
| I-38 | 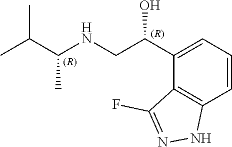 | 266.3 |
| I-39 | 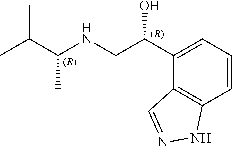 | 234.3 |
| I-40 | 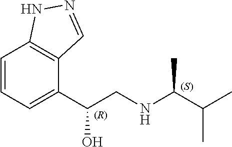 | 248.3 |
| I-41 | 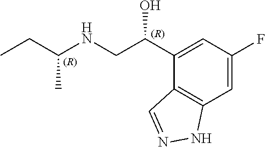 | 252.3 |
| I-42 | 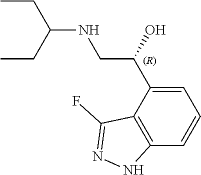 | 266.3 |
| I-43 | 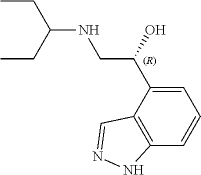 | 248.3 |
| I-44 | 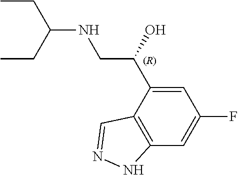 | 266.3 |
| I-45 | 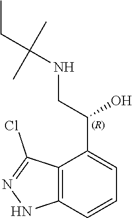 | 282.7 |
| I-46 | 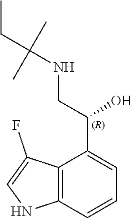 | 265.3 |
| I-47 | 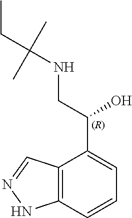 | 248.4 |
| I-48 | 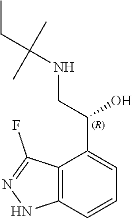 | 266.3 |
| I-49 | 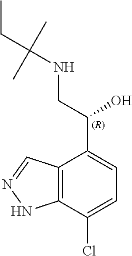 | 282.8 |
| I-50 | 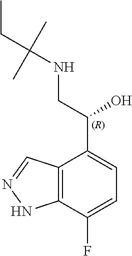 | 266.3 |
| I-51 | 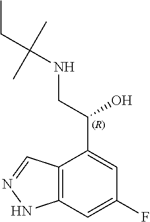 | 266.3 |
| I-52 | 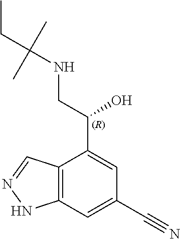 | 273.4 |
| 1-53 | 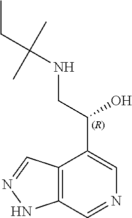 | 249.3 |
| I-54 | 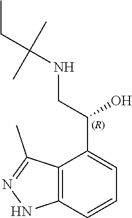 | 262.4 |

In some embodiments, the present disclosure provides a compound set forth in Table 1A, above, or a pharmaceutically acceptable salt thereof.

TABLE 1B
Exemplary compounds

| | | MS |
|---|---|---|
| I-# | Structure | (M + H) |
| I-55 | 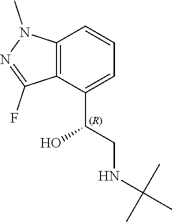 | 266.3 |
| I-56 | 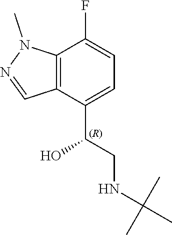 | 266.3 |
| I-57 | 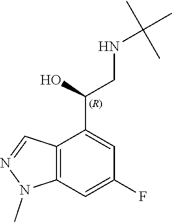 | 266.3 |
| I-58 | 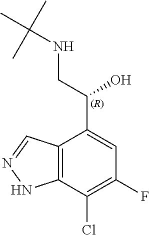 | 286.8 |
| I-59 | 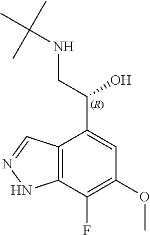 | 282.3 |
| I-60 | 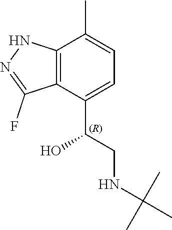 | 266.3 |
| I-61 | 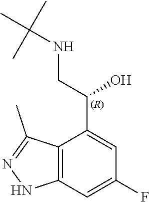 | 266.3 |
| I-62 | 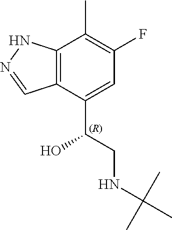 | 266.3 |

In some embodiments, the present disclosure provides a compound set forth in Table 1B, above, or a pharmaceutically acceptable salt thereof.

TABLE 1C
Exemplary compounds

| | | MS |
|---|---|---|
| I-# | Structure | (M + H) |
| I-63 | 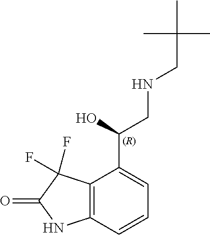 | 299.3 |
| I-64 | 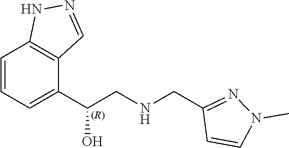 | 272.2 |
| I-65 | 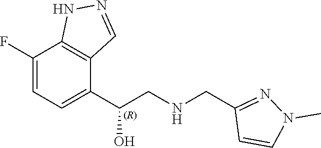 | 290.3 |
| I-66 | 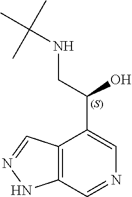 | 235.3 |
| I-67 | 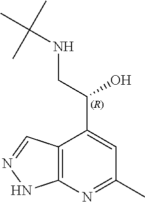 | 249.2 |
| I-68 | 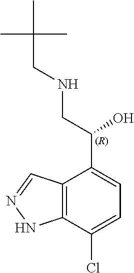 | 282.7 |
| I-69 | 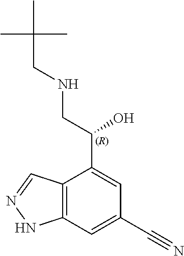 | 273.3 |
| I-70 | 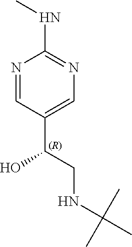 | 225.3 |
| I-71 | 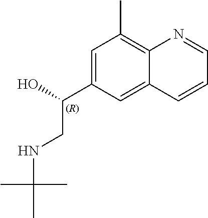 | 259.4 |
| I-72 | 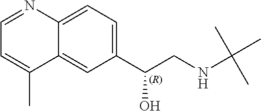 | 259.4 |
| I-73 | 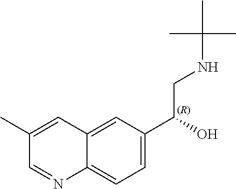 | 259.4 |
| I-74 | 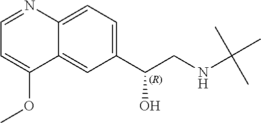 | 275.3 |
| I-75 | 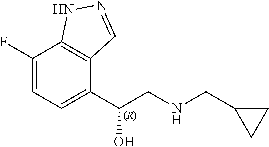 | 250.3 |
| I-76 | 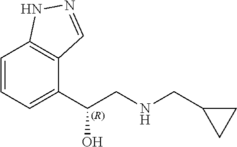 | 232.4 |
| I-77 | 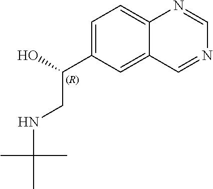 | 246.4 |
| I-78 | 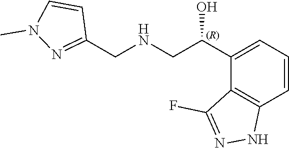 | 290.3 |
| I-79 | 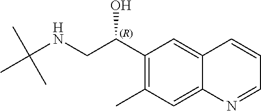 | 259.4 |
| I-80 | 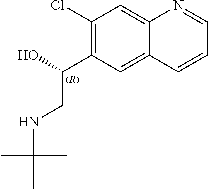 | 279.8 |
| I-81 | 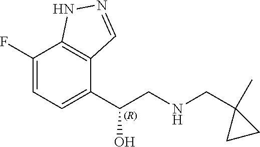 | 264.3 |
| I-82 | 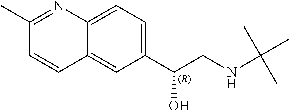 | 259.4 |
| I-83 | 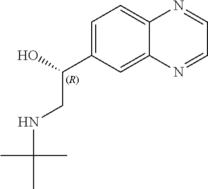 | 246.3 |
| I-84 | 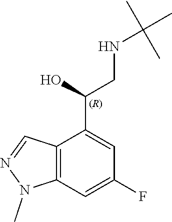 | 266.3 |
| I-85 | 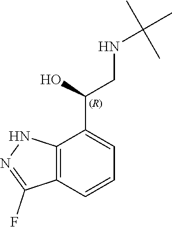 | 252.3 |
| I-86 | 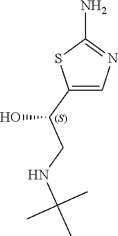 | 216.2 |
| I-87 | 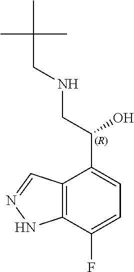 | 266.3 |
| I-88 | 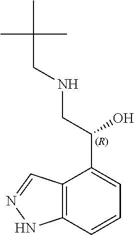 | 248.3 |
| I-89 | 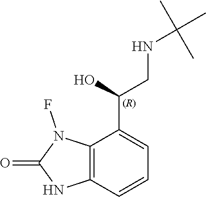 | 267.3 |
| I-90 | 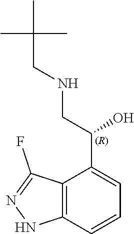 | 266.3 |
| I-91 | 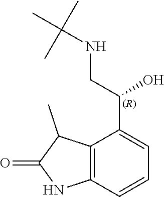 | 263.4 |
| I-92 | 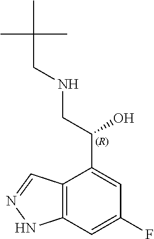 | 266.3 |
| I-93 | 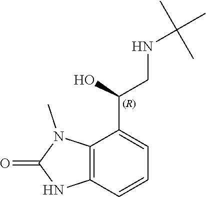 | 264.3 |
| I-94 | 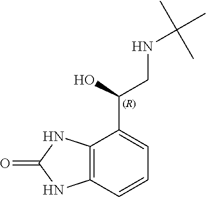 | 250.3 |
| I-95 | 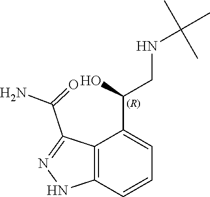 | 277.4 |
| I-96 | 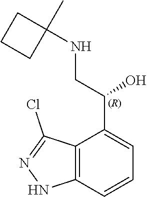 | 280.7 |
| I-97 | 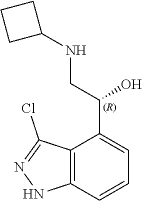 | 266.7 |
| I-98 | 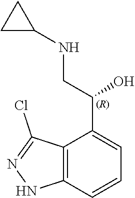 | 252.8 |
| I-99 | 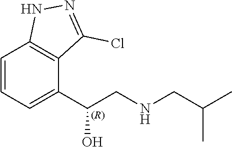 | 268.7 |
| I-100 | 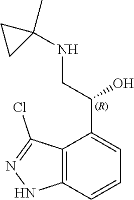 | 266.7 |
| I-101 | 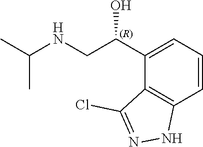 | 254.7 |
| I-102 | 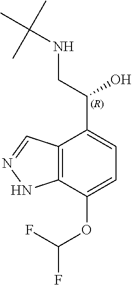 | 300.3 |
| I-103 | 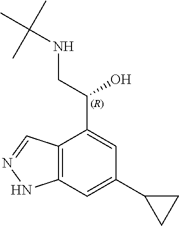 | 274.4 |
| I-104 | 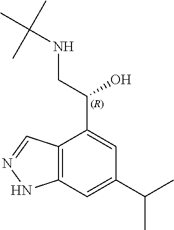 | 276.4 |
| I-105 | 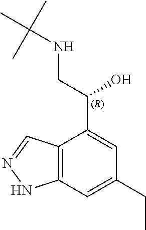 | 262.4 |
| I-106 | 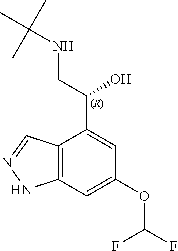 | 300.4 |
| I-107 | 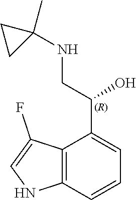 | 249.3 |
| I-108 | 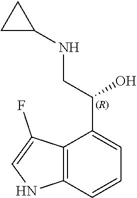 | 235.3 |
| I-109 | 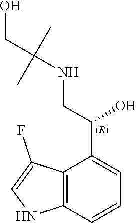 | 267.3 |
| I-110 | 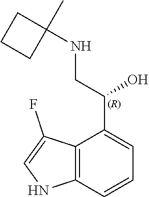 | 263.4 |
| I-111 | 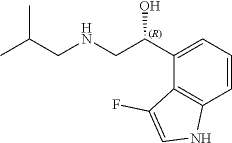 | 251.3 |
| I-112 | 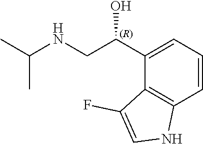 | 237.4 |
| I-113 | 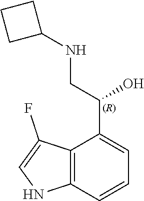 | 249.3 |
| I-114 | 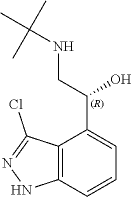 | 268.8 |
| I-115 | 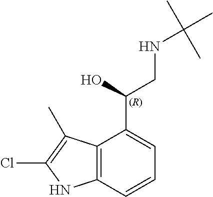 | 281.9 |
| I-116 | 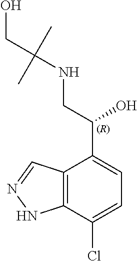 | 284.8 |
| I-117 | 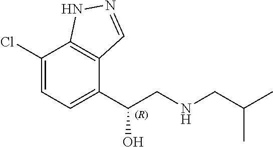 | 268.8 |
| I-118 | 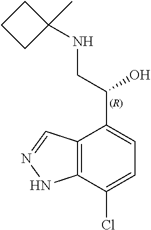 | 280.7 |
| I-119 | 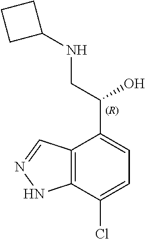 | 266.7 |
| I-120 | 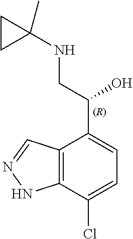 | 266.7 |
| I-121 | 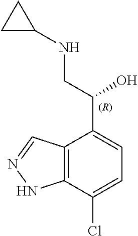 | 252.8 |
| I-122 | 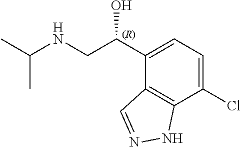 | 254.7 |
| I-123 | 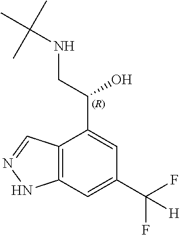 | 284.3 |
| I-124 | 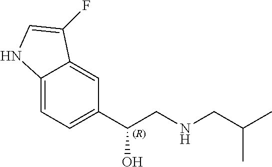 | 251.4 |
| I-125 | 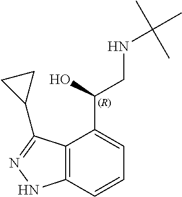 | 274.4 |
| I-126 | 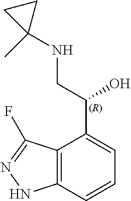 | 250.4 |
| I-127 | 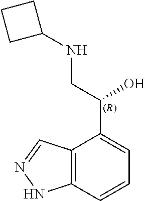 | 232.3 |
| I-128 | 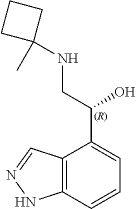 | 246.3 |
| I-129 | 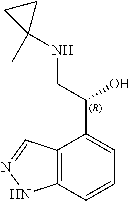 | 232.4 |
| I-130 | 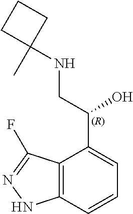 | 264.3 |
| I-131 | 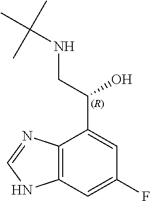 | 252.3 |
| I-132 | 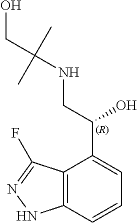 | 268.3 |
| I-133 | 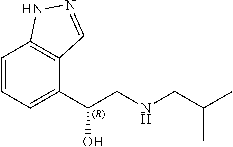 | 234.3 |
| I-134 | 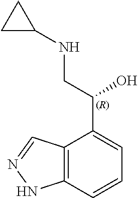 | 218.3 |
| I-135 | 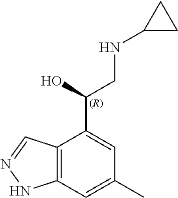 | 232.4 |
| I-136 | 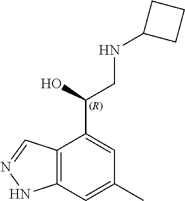 | 246.4 |
| I-137 | 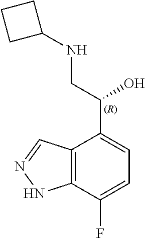 | 250.3 |
| I-138 | 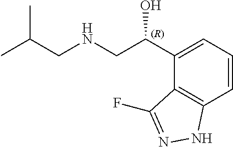 | 252.4 |
| I-139 | 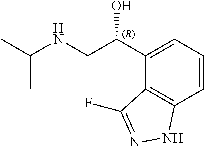 | 238.3 |
| I-140 | 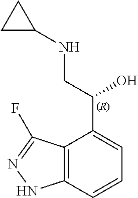 | 236.3 |
| I-141 | 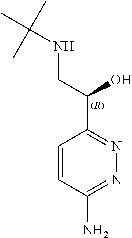 | 211.2 |
| I-142 | 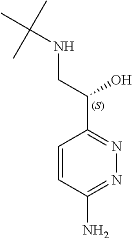 | 211.2 |
| I-143 | 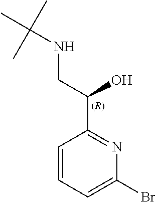 | 274.2 |
| I-144 | 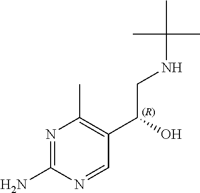 | 225.3 |
| I-145 | 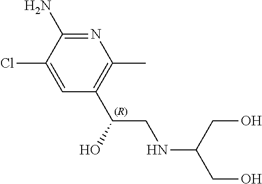 | 276.7 |
| I-146 | 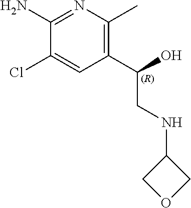 | 258.6 |
| I-147 | 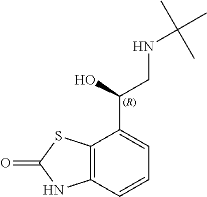 | 267.4 |
| I-148 | 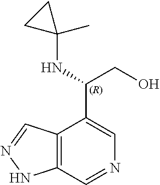 | 233.4 |
| I-149 | 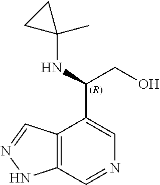 | 233.4 |
| I-150 | 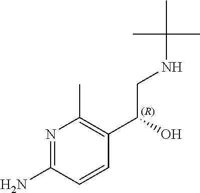 | 224.3 |
| I-151 | 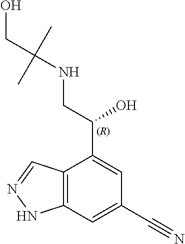 | 275.3 |
| I-152 | 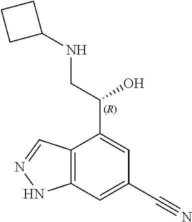 | 257.4 |
| I-153 | 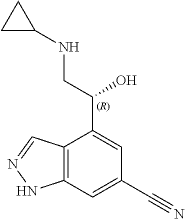 | 243.2 |
| I-154 | 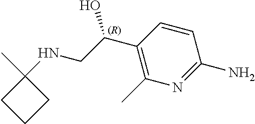 | 236.4 |
| I-155 | 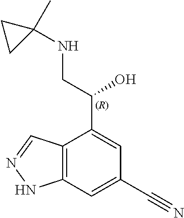 | 257.3 |
| I-156 | 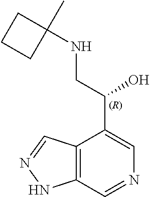 | 247.3 |
| I-157 | 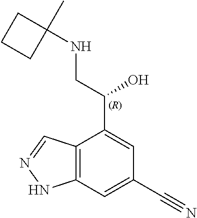 | 271.3 |
| I-158 | 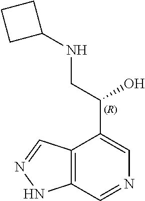 | 233.2 |
| I-159 | 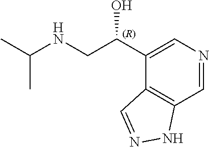 | 221.3 |
| I-160 | 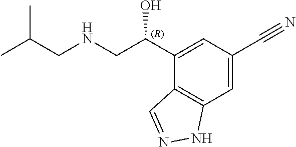 | 259.2 |
| I-161 | 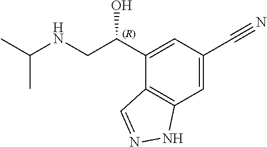 | 245.2 |
| I-162 | 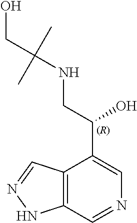 | 251.3 |
| I-163 | 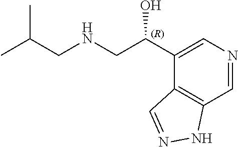 | 235.4 |
| I-164 | 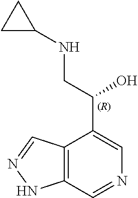 | 219.4 |
| I-165 | 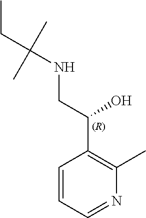 | 223.3 |
| I-166 | 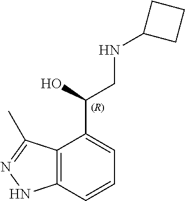 | 246.4 |
| I-167 | 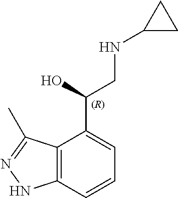 | 232.4 |
| I-168 | 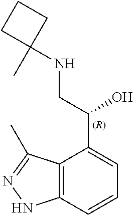 | 260.3 |
| I-169 | 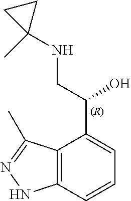 | 246.4 |
| I-170 | 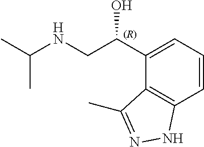 | 234.3 |
| I-171 | 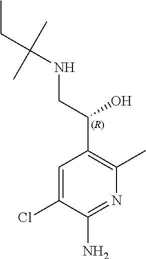 | 272.8 |
| I-172 | 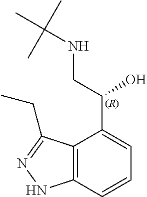 | 262.3 |
| I-173 | 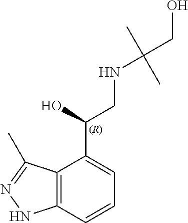 | 264.3 |
| I-174 | 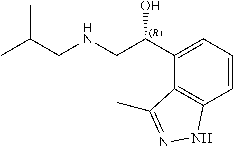 | 248.3 |
| I-175 | 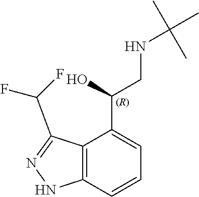 | 284.3 |
| I-176 | 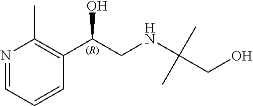 | 225.3 |
| I-177 | 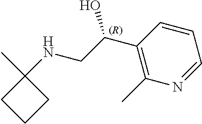 | 221.4 |
| I-178 | 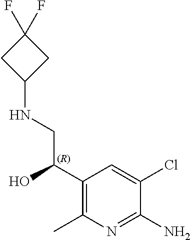 | 292.8 |
| I-179 | 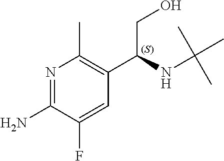 | 242.3 |
| I-180 | 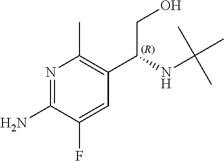 | 242.3 |
| I-181 | 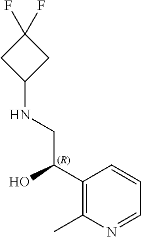 | 243.2 |
| I-182 | 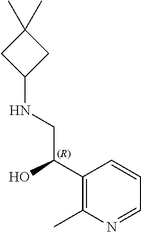 | 235.3 |
| I-183 | 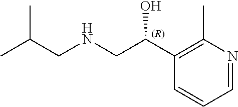 | 209.3 |
| I-184 | 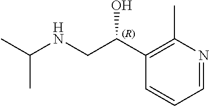 | 195.4 |
| I-185 | 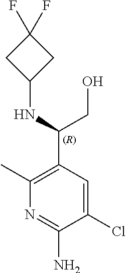 | 292.7 |
| I-186 | 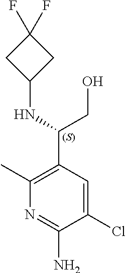 | 292.7 |
| I-187 | 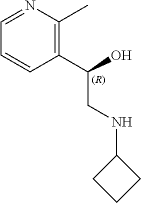 | 207.2 |
| I-188 | 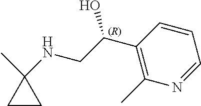 | 207.2 |
| I-189 | 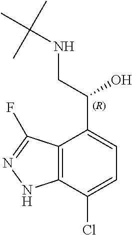 | 286.7 |

In some embodiments, the present disclosure provides a compound set forth in Table 1C, above, or a pharmaceutically acceptable salt thereof.

### General Methods of Providing the Present Compounds

The compounds of this disclosure may be prepared or isolated in general by synthetic and/or semi-synthetic methods known to those skilled in the art for analogous compounds and by methods described in detail in the Examples, herein.

In the Schemes below, where a particular protecting group, leaving group, or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

As used herein, the phrase “oxygen protecting group” includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

The compounds of the present disclosure can be prepared in a variety of fashions. In some embodiments, compounds of the present disclosure are generally prepared according to Scheme 1 set forth below:

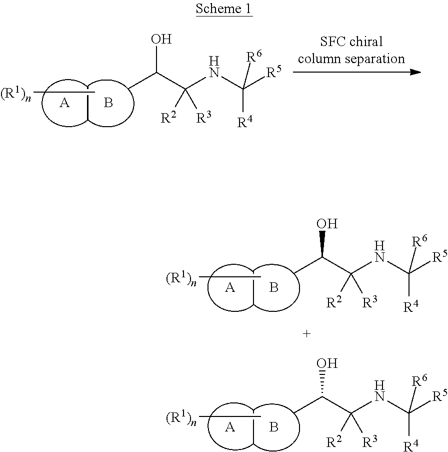

In some embodiments, compounds of the present disclosure are generally prepared according to Scheme 2 set forth below:

In some embodiments, compounds of the present disclosure are generally prepared according to Scheme 3 set forth below:

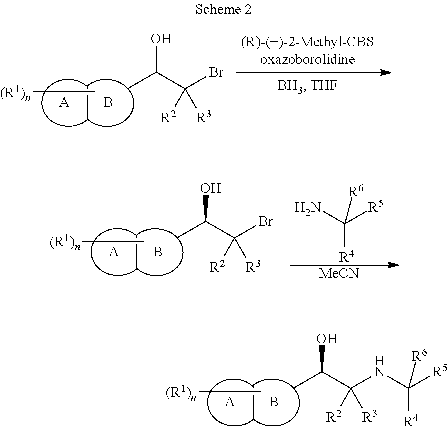

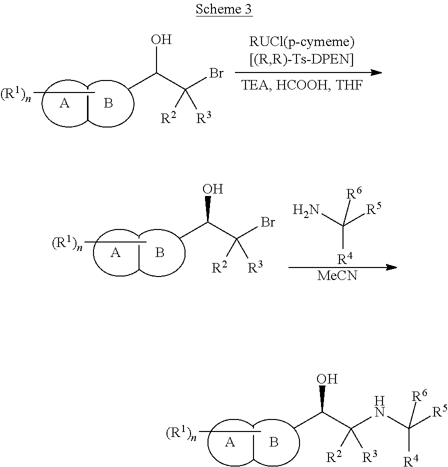

In some embodiments, compounds of the present disclosure are generally prepared according to Scheme 4 set forth below:

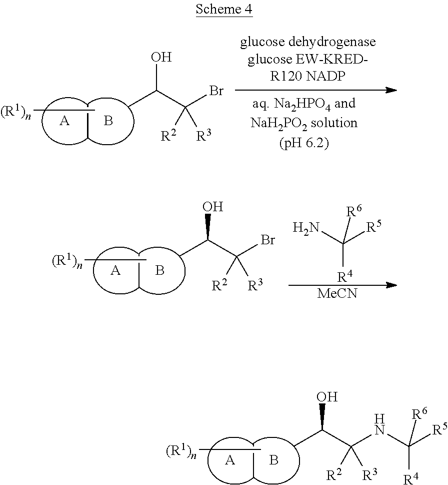

In some embodiments, compounds of the present disclosure are generally prepared according to Scheme 5 set forth below:

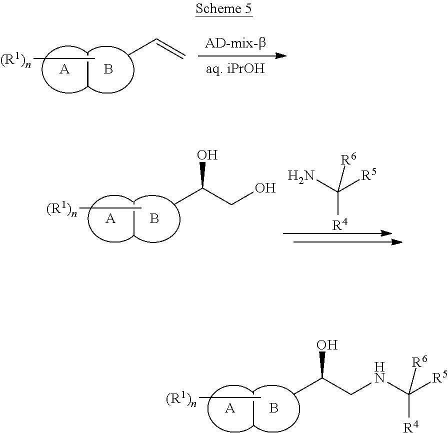

In some embodiments, compounds of the present disclosure are generally prepared according to Scheme 6 set forth below:

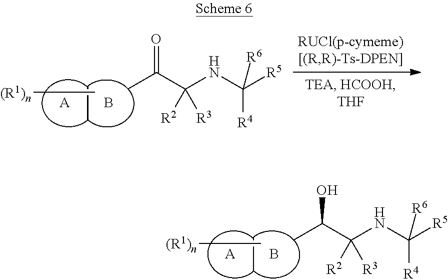

In some embodiments, compounds of the present disclosure are generally prepared according to Scheme 7 set forth below:

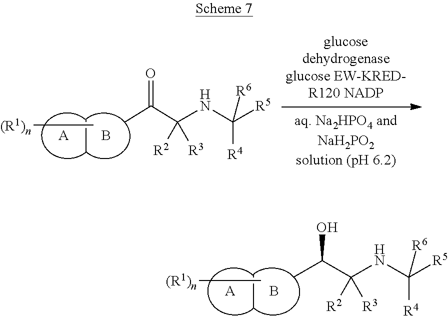

One of skill in the art will appreciate that various functional groups present in compounds of the disclosure such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. See for example, “March's Advanced Organic Chemistry”, 5^{th} Ed., Ed.: Smith, M. B. and March, J., John Wiley & Sons, New York: 2001, the entirety of each of which is herein incorporated by reference. Such interconversions may require one or more of the aforementioned techniques, and certain methods for synthesizing compounds of the disclosure are described below in the Exemplification.

### Pharmaceutical Compositions of the Present Compounds

According to another embodiment, the disclosure provides a composition comprising a compound of this disclosure or a pharmaceutically acceptable derivative thereof and a pharmaceutically acceptable carrier, adjuvant, or vehicle. The amount of compound in compositions of this disclosure is such that is effective to measurably agonize a beta adrenergic receptor or a mutant thereof, in a biological sample or in a subject. In certain embodiments, the amount of compound in compositions of this disclosure is such that is effective to measurably agonize a beta adrenergic receptor or a mutant thereof, in a biological sample or in a subject. In certain embodiments, a composition of this disclosure is formulated for administration to a subject in need of such composition. In preferred embodiments, a composition of this disclosure is formulated for oral administration to a subject.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In some embodiments, the pharmaceutically acceptable carriers, adjuvants or vehicles comprises granules, enteric coatings, capsules, conventional tablets, multilayer tablets, controlled-release agents, effervescents, orally administered tables, or orally administered films, which are discussed in sections below.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily active metabolite or residue thereof.

As used herein, the term “inhibitorily active metabolite or residue thereof” means that a metabolite or residue thereof is also a modulator of a beta adrenergic receptor or a mutant thereof.

In some embodiment, the compositions of this disclosure are formulated for pharmaceutical administration to a subject or patient, e.g., a mammal, preferably a human being. Such pharmaceutical compositions are used to ameliorate, treat or prevent any of the diseases described herein in a subject.

Agents of the disclosure are often administered as pharmaceutical compositions comprising an active therapeutic agent, i.e., and a variety of other pharmaceutically acceptable components. See Remington's Pharmaceutical Science (15th ed., Mack Publishing Company, Easton, Pa., 1980). The preferred form depends on the intended mode of administration and therapeutic application. The compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In some embodiments, the present disclosure provides pharmaceutically acceptable compositions comprising a therapeutically effective amount of one or more of a described compound, formulated together with one or more pharmaceutically acceptable carriers (additives) and/or diluents for use in treating the diseases described herein, including, but not limited to stroke, ischemia, Alzheimer's, ankylosing spondylitis, arthritis, osteoarthritis, rheumatoid arthritis, psoriatic arthritis, asthma atherosclerosis, Crohn's disease, colitis, dermatitis diverticulitis, fibromyalgia, hepatitis, irritable bowel syndrome, systemic lupus erythematous, nephritis, ulcerative colitis and Parkinson's disease. While it is possible for a described compound to be administered alone, it is preferable to administer a described compound as a pharmaceutical formulation (composition) as described herein. The provided compounds may be formulated for administration in any convenient way for use in human or veterinary medicine, by analogy with other pharmaceuticals.

As described in detail, pharmaceutical compositions of the present disclosure may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations for use in accordance with the present disclosure include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient, which can be combined with a carrier material, to produce a single dosage form will vary depending upon the host being treated, and the particular mode of administration. The amount of active ingredient that can be combined with a carrier material to produce a single dosage form will generally be that amount of the compound, which produces a therapeutic effect. Generally, this amount will range from about 1% to about 99% of active ingredient. In some embodiments, this amount will range from about 5% to about 70%, from about 10% to about 50%, or from about 20% to about 40%.

In certain embodiments, a formulation as described herein comprises an excipient selected from cyclodextrins, liposomes, micelle forming agents, e.g., bile acids, and polymeric carriers, e.g., polyesters and polyanhydrides, and a provided compound. In certain embodiments, an aforementioned formulation renders a provided compound or pharmaceutically acceptable salt thereof orally bioavailable.

Methods of preparing formulations or compositions comprising a provided compound include a step of bringing into association a provided compound with the carrier and, optionally, one or more accessory ingredients. In general, formulations may be prepared by uniformly and intimately bringing into association a provided compound with liquid carriers, or finely divided solid carriers, or both, and then, if necessary, shaping the product.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as those described in Pharmacopeia Helvetica, or a similar alcohol. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In some cases, in order to prolong the effect of a drug, it may be desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of a provided compound in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

The pharmaceutical compositions of this disclosure may be orally administered in any orally acceptable dosage form including, but not limited to, capsules, tablets, and aqueous suspensions and solutions. In the case of tablets for oral use, carriers, which are commonly used include lactose and corn starch. Lubricating agents, such as magnesium stearate, are also typically added. For oral administration in a capsule form, useful diluents include lactose and dried cornstarch. When aqueous suspensions and solutions and propylene glycol are administered orally, a provided compound may be combined with emulsifying and suspending agents. If desired, certain sweetening and/or flavoring and/or coloring agents may be added.

Formulations described herein suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a provided compound or pharmaceutically acceptable salt thereof as an active ingredient. A provided compound may also be administered as a bolus, electuary or paste.

In solid dosage forms for oral administration (capsules, tablets, pills, dragees, powders, granules and the like), an active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

Tablets may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent. If a solid carrier is used, the preparation can be in tablet form, placed in a hard gelatin capsule in powder or pellet form, or in the form of a troche or lozenge. The amount of solid carrier will vary, e.g., from about 25 to 800 mg, preferably about 25 mg to 400 mg. When a liquid carrier is used, the preparation can be, e.g., in the form of a syrup, emulsion, soft gelatin capsule, sterile injectable liquid such as an ampule or nonaqueous liquid suspension. Where the composition is in the form of a capsule, any routine encapsulation is suitable, for example, using the aforementioned carriers in a hard gelatin capsule shell.

Tablets and other solid dosage forms, such as dragees, capsules, pills and granules, may optionally be scored or prepared with coatings and shells, such as enteric coatings and other coatings well known in the pharmaceutical-formulating art. They may alternatively or additionally be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of a provided compound include pharmaceutically acceptable emulsions, microemulsions, solutions, suspensions, syrups and elixirs. In addition a provided compound, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, oral compositions can also include adjuvants such as wetting agents, emulsifying and suspending agents, sweetening, flavoring, coloring, perfuming and preservative agents.

Suspensions, in addition to active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The pharmaceutical compositions of the present disclosure may also be administered in the form of suppositories for rectal administration. These compositions can be prepared by mixing a provided compound with a suitable non-irritating excipient, which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this disclosure is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this disclosure include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this disclosure may also be topically applied to the lower intestinal tract by rectal suppository formulation or in a suitable enema formulation. Topically-administered transdermal patches are also included in this disclosure.

The pharmaceutical compositions of this disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

Transdermal patches have the added advantage of providing controlled delivery of a provided compound to the body. Dissolving or dispersing the compound in the proper medium can make such dosage forms. Absorption enhancers can also be used to increase the flux of the compound across the skin. Either providing a rate controlling membrane or dispersing the compound in a polymer matrix or gel can control the rate of such flux.

Examples of suitable aqueous and nonaqueous carriers, which may be employed in the pharmaceutical compositions of the disclosure, include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Such compositions may also contain adjuvants such as preservatives, wetting agents, emulsifying agents and dispersing agents. Inclusion of one or more antibacterial and/or antifungal agents, for example, paraben, chlorobutanol, phenol sorbic acid, and the like, may be desirable in certain embodiments. It may alternatively or additionally be desirable to include isotonic agents, such as sugars, sodium chloride, and the like into the compositions. In addition, prolonged absorption of the injectable pharmaceutical form may be brought about by the inclusion of agents, which delay absorption such as aluminum monostearate and gelatin.

In certain embodiments, a described compound or pharmaceutical preparation is administered orally. In other embodiments, a described compound or pharmaceutical preparation is administered intravenously. Alternative routes of administration include sublingual, intramuscular, and transdermal administrations.

When compounds described herein are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

Preparations described herein may be given orally, parenterally, topically, or rectally. They are of course given in forms suitable for the relevant administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

Such compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, compounds described herein which may be used in a suitable hydrated form, and/or the pharmaceutical compositions of the present disclosure, are formulated into pharmaceutically-acceptable dosage forms by conventional methods known to those of skill in the art.

Actual dosage levels of the active ingredients in the pharmaceutical compositions of the disclosure may be varied so as to obtain an amount of the active ingredient that is effective to achieve the desired therapeutic response for a particular patient, composition, and mode of administration, without being toxic to the patient.

Also provided are kits that include a provided adrenergic receptor modulating compounds. Systems of the present disclosure include collections of active agents brought together, e.g., by a health care practitioner, for administration to a subject, such as a patient. Such systems may include a provided adrenergic receptor modulating compound and one or more additional active agents disclosed herein. Kits that include adrenergic receptor modulating compounds which are provided that may include one or more dosages of a provided adrenergic receptor modulating compound, and optionally one or more dosages of one or more additional active agents. Conveniently, the formulations may be provided in a unit dosage format. In such kits, in addition to the containers containing the formulation(s), e.g. unit doses, is an informational package insert describing the use of the subject formulations in the methods of the as disclosed herein, e.g., instructions for using the subject unit doses to CNS disease conditions. These instructions may be present in the subject systems and kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

Most preferably, pharmaceutically acceptable compositions of this disclosure are formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of compound of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

The compounds utilized in the compositions and methods of this disclosure may also be modified by appending appropriate functionalities to enhance selective biological properties. Such modifications are known in the art and include those, which increase biological penetration into a given biological system (e.g., blood, lymphatic system, or central nervous system), increase oral availability, increase solubility to allow administration by injection, alter metabolism and/or alter rate of excretion.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

### Uses of the Present Pharmaceutical Compositions and Compounds in Therapy

Compounds and compositions described herein are generally useful for the modulation of an adrenergic receptor.

As used herein, the term “adrenergic receptor-mediated” disorders, diseases, and/or conditions as used herein means any disease or other deleterious condition in which adrenergic receptors are known to play a role. Accordingly, another embodiment of the present disclosure relates to treating or lessening the severity of one or more diseases in which adrenergic receptors or a mutant thereof, is known to play a role.

In some embodiments, the present disclosure provides a method of treating a subject with an adrenergic receptor-mediated disorder, disease, and/or condition, the method including administering to the subject a therapeutically effective amount of a provided compound or pharmaceutically acceptable salt thereof. In some embodiments, the adrenergic receptor-mediated disorder, disease, and/or condition is a disease associated with a beta-adrenergic receptor. In some embodiments, the adrenergic receptor-mediated disorder, disease, and/or condition is a neurodegenerative disease. In some embodiments, the subject is a human.

In some embodiments, a compound as disclosed herein may be an adrenergic receptor modulating compound (e.g., an agonist, partial agonist or antagonist of an adrenergic receptor). The adrenergic receptor modulating compounds of the present disclosure can in some embodiments find use in modulating the activity of a target adrenergic receptor in vitro or in vivo. Aspects of the subject methods include contacting a sample with an effective amount of an adrenergic receptor modulating compound (e.g., as described herein) to determine whether the activity desired exists.

Adrenergic receptors (ADRs) are G-protein coupled receptors (GPCR) that are widely expressed throughout the body and play an important role in regulating multiple physiological processes including cognition, stress-related behavior, inflammation, and smooth muscle contraction/dilation, cardiac muscle contraction, airway reactivity and cognition. Adrenergic receptors mediate the central and peripheral effects of noradrenaline (NA) and adrenaline. Multiple subtypes of ADRs exist, including α-adrenergic receptors and β-adrenergic receptors. Each subtype is expressed in distinct patterns and involved in different physiological processes. Therefore, ligands that selectively target one subtype are valuable both as research tools to identify the roles of different ADR subtypes and as therapeutic agents for multiple diseases related to dysfunction of the NA and adrenaline systems.

β-adrenergic receptors further include three sub-types: β1-adrenergic receptor (β1-ADR), β2-adrenergic receptor (β2-ADR), and β3-adrenergic receptor (β3-ADR). Because these subtypes are expressed in distinct patterns and involved in different physiological processes, ligands that can selectively target one subtype have therapeutic potential for multiple diseases. However, discovery of subtype-selective ligands has been challenging due to a high level of sequence homology shared by these subtypes. A lot of existing agonists for β-adrenergic receptors also exhibit inferior blood-brain-barrier (BBB) penetration, which is required in an effort for drug discovery for central nervous system (CNS) indications.

As a class of G-protein coupled receptor, adrenergic receptors signal via G protein- and β-arrestin-dependent pathways. G protein or β-arrestin signaling can mediate different physiological responses. Recently, it has become clear that agonists can show biased activation of signaling pathways. The ability of ligands to activate the receptor and produce responses in a pathway-dependent manner has been termed “signaling bias” or “functional selectivity”. As G proteins and β-arrestins mediate distinct physiological processes, biased agonists can provide improved therapeutic selectivity with reduced adverse effects. Thus, in some embodiments, the present disclosure is directed to 0-adrenergic receptor subtype-selective agonists with improved blood-brain-barrier (BBB) penetration.

In some embodiments, a provided compound is an adrenergic receptor modulating compound and can be an agonist of the target adrenergic receptor. In some cases, an effective amount of a provided adrenergic receptor modulating compound is an amount sufficient to activate an activity related to the adrenergic receptor in a cell by 10% or more, such as 20% or more, 30% or more, 40% or more, 50% or more, 60% or more, 70% or more, 80% or more, 90% or more, 100% or more, 200% or even more relative to a control, e.g., a control cell exhibiting a known activity level of the receptor.

In some embodiments, a provided compound is an adrenergic receptor modulating compound and can be a partial agonist of the target adrenergic receptor. In some cases, an effective amount of a provided adrenergic receptor modulating compound is an amount sufficient to achieve partially agonism of the adrenergic receptor in a cell, e.g., where the subject compound achieves 10% activation or more of the receptor, such as 20% or more, 30% or more, 40% or more, 50% or more, 60% or more, 70% or more, 80% or more, or 90% or more, relative to a control, e.g., a receptor that is fully activated. Partial agonism may be assessed using any convenient methods, such as a cell based assay using a known full agonist as a 100% activation control, where the relative maximum activation of the receptor can be measured relative to the full agonist.

In some embodiments, a provided compound is an adrenergic receptor modulating compound and can be an antagonist of the target adrenergic receptor. In some cases, an effective amount of a provided adrenergic receptor modulating compound is an amount sufficient to inhibit or decrease the activity of the target adrenergic receptor in a sample by 10% or more, such as 20% or more, 30% or more, 40% or more, 50% or more, 60% or more, 70% or more, 80% or more, 90% or more, or even more relative to a control, e.g., a sample not contacted with the compound of interest.

In some embodiments, a provided compound acts as a low nM partial agonist of the β2 adrenergic receptor. For instance, in some embodiments, a provided compound has an EC₅₀ of less than about 1 nM, less than about 5 nM, less than about 10 nM, less than about 15 nM, less than about 20 nM, less than 25 nM, less than 30 nM, less than 35 nM, less than 40 nM, less than 45 nM, less than 50 nM, less than 55 nM, less than 60 nM, less than 65 nM, less than 70 nM, less than 75 nM, less than 80 nM, less than 85 nM, less than 90 nM, less than 95 nM, or less than 100 nM. In some embodiments, a provided compound acts as a low nM partial agonist of the β2 adrenergic receptor and has an EC₅₀ of from about 0.001 nM to about 200 nM, 0.001 nM to about 150 nM, about 0.001 nM to about 100 nM, 0.01 nM to about 100 nM, 0.1 nM to about 100 nM, or about 0.1 nM to about 80 nM, or about 0.1 nM to about 60 nM, or about 0.1 nM to about 40 nM, or about 0.1 nM to about 30 nM, or about 0.1 nM to about 20 nM, or about 0.1 nM to about 10 nM.

In some embodiments, a provided compound acts as a low M partial agonist of the β2 adrenergic receptor. For instance, in some embodiments, a provided compound has an EC₅₀ of less than about 0.1 μM, less than about 0.5 μM, less than about 1.0 μM, less than about 1.5 μM, less than about 2.0 μM, less than about 2.5 μM, less than about 3.0 μM, less than about 3.5 μM, less than about 4.0 μM, less than about 4.5 μM, less than about 5.0 μM, less than about 5.5 μM, less than about 6.0 μM, less than about 6.5 μM, less than about 7.0 μM, less than about 7.5 μM, less than about 8.0 μM, less than about 8.5 μM, less than about 9.0 μM, less than about 9.5 μM, or less than about 10.0 μM,

In some embodiments, a provided compound acts as a low M partial agonist of the β2 adrenergic receptor and has an EC₅₀ of from about 0.01 μM to about 10 μM, about 0.01 μM to about 9.0 μM, about 0.01 μM to about 8.0 μM, about 0.01 μM to about 7.0 μM, about 0.01 μM to about 6.0 μM, about 0.01 μM to about 5.0 μM, about 0.01 μM to about 4.0 μM, about 0.01 μM to about 3.0 μM, about 0.01 M to about 2.0 μM, about 0.01 M to about 1.0 μM, about 0.01 M to about 9.0 μM, about 0.1 μM to about 1.0 μM,

In some embodiments, the present disclosure provides a method of treating a subject with a adrenergic receptor-mediated disorder, disease, and/or condition, wherein the target adrenergic receptor is a β1-adrenergic receptor. In some embodiments of the method, the target adrenergic receptor is a β2-adrenergic receptor. In some embodiments of the method, the target adrenergic receptor is a β3-adrenergic receptor. In some embodiments, the compound is an agonist for both β1-adrenergic receptor and β2-adrenergic receptor. In certain cases, the compound is selective for the β2-adrenergic receptor over a β1-adrenergic receptor.

In some embodiments, the target adrenergic receptor may be one that is responsible for a mediating an intracellular signal or pathway in a cell. In some embodiments, the sample includes a cell and modulating the adrenergic receptor modulates a physiological process in the cell. Any convenient physiological processes can be targeted for modulation in a cell using the subject methods. In some embodiments, the physiological process is one that is implicated in cardiac function, in certain instances, the physiological process is one that is implicated in cognitive function. In certain instances, the physiological process is one that is implicated in an inflammatory pathway or condition. The subject methods can provide for mediation of the intracellular concentration of a signaling molecule in a cell, such as cAMP. The subject methods can provide for partial or full blockage of the target adrenergic receptor to result in modulation (e.g., activation) of cAMP in a sample. In some embodiments, the method does not modulate β-arrestin pathways of the cell. In some cases, the cells are inflammatory cells and the function of the cells is regulated. The subject methods can provide for inhibition of an inflammatory pathway in a cell. In some cases, TNF-alpha is inhibited in the cell, e.g., the concentration or production of TNF-alpha is reduced by practicing the subject method. In certain embodiments of the method, the cell is a neuron. In some embodiments, modulating the adrenergic receptor enhances neurogenesis.

In some embodiments, a provide compound or a pharmaceutically acceptable salt thereof may be employed in a conventional manner for controlling, preventing, treating a disease described herein, including, but not limited to, myocardial infarction, stroke, ischemia, Alzheimer's disease, Parkinson's disease, Gehrig's disease (Amyotrophic Lateral Sclerosis), Huntington's disease, Multiple Sclerosis, senile dementia, subcortical dementia, arteriosclerotic dementia, AIDS-associated dementia, other dementias, cerebral vasculitis, epilepsy, Tourette's syndrome, Wilson's disease, Pick's disease, encephalitis, encephalomyelitis, meningitis, prion diseases, cerebellar ataxias, cerebellar degeneration, spinocerebellar degeneration syndromes, Friedrich's ataxia, ataxia telangiectasia, spinal dysmyotrophy, progressive supranuclear palsy, dystonia, muscle spasticity, tremor, retinitis pigmentosa, striatonigral degeneration, mitochondrial encephalomyopathies, neuronal ceroid lipofuscinosis, cerebral autosomal dominant arteriopathy with subcortical infarcts (CADASIL) and diabetic retinopathy. Such methods of treatment, their dosage levels and requirements may be selected by those of ordinary skill in the art from available methods and techniques.

In some embodiments, the disease is selected from myocardial infarction, stroke, ischemia, Alzheimer's disease, Parkinson's disease, Gehrig's disease (Amyotrophic Lateral Sclerosis), Huntington's disease, Multiple Sclerosis, senile dementia, subcortical dementia, arteriosclerotic dementia, AIDS-associated dementia, other dementias, cerebral vasculitis, epilepsy, Tourette's syndrome, Wilson's disease, Pick's disease, encephalitis, encephalomyelitis, meningitis, prion diseases, cerebellar ataxias, cerebellar degeneration, spinocerebellar degeneration syndromes, Friedrich's ataxia, ataxia telangiectasia, spinal dysmyotrophy, progressive supranuclear palsy, dystonia, muscle spasticity, tremor, retinitis pigmentosa, striatonigral degeneration, mitochondrial encephalomyopathies, and neuronal ceroid lipofuscinosis.

In some embodiments, the disease is a neurodegenerative disease that is one or more selected from MCI (mild cognitive impairment), aMCI (amnestic MCI), Vascular Dementia, Mixed Dementia, FTD (fronto-temporal dementia; Pick's disease), HD (Huntington disease), Rett Syndrome, PSP (progressive supranuclear palsy), CBD (corticobasal degeneration), SCA (spinocerebellar ataxia), MSA (Multiple system atrophy), SDS (Shy-Drager syndrome), olivopontocerebellar atrophy, TBI (traumatic brain injury), CTE (chronic traumatic encephalopathy), stroke, WKS (Wernicke-Korsakoff syndrome; alcoholic dementia & thiamine deficiency), normal pressure hydrocephalus, hypersomnia/narcolepsy, ASD (autistic spectrum disorders), FXS (fragile X syndrome), TSC (tuberous sclerosis complex), prion-related diseases (CJD etc.), depressive disorders, DLB (dementia with Lewy bodies), PD (Parkinson's disease), PDD (PD dementia), ADHD (attention deficit hyperactivity disorder), Alzheimer's disease (AD), early AD, and Down Syndrome (DS). In some embodiments the disease is a neurodegenerative disease that is one or more selected from the group consisting of MCI, aMCI, Vascular Dementia, Mixed Dementia, FTD (fronto-temporal dementia; Pick's disease), HD (Huntington disease), Rett Syndrome, PSP (progressive supranuclear palsy), CBD (corticobasal degeneration), SCA (spinocerebellar ataxia), MSA (Multiple system atrophy), SDS (Shy-Drager syndrome), olivopontocerebellar atrophy, TBI (traumatic brain injury), CTE (chronic traumatic encephalopathy), stroke, WKS (Wernicke-Korsakoff syndrome; alcoholic dementia & thiamine deficiency), normal pressure hydrocephalus, hypersomnia/narcolepsy, ASD (autistic spectrum disorders), FXS (fragile X syndrome), TSC (tuberous sclerosis complex), prion-related diseases (CJD etc.), depressive disorders, DLB (dementia with Lewy bodies), PD (Parkinson's disease), PDD (PD dementia), and ADHD (attention deficit hyperactivity disorder). In some embodiments the subject does not have Alzheimer's disease (AD). In some embodiments the subject does not have Down Syndrome.

In some embodiments, the present disclosure provides a method of treating a subject with a beta-adrenergic receptor-mediated disorder, disease, and/or condition, the method comprising administering to the subject a provided compound or a pharmaceutically acceptable salt thereof and a peripherally acting β-blocker (PABRA).

As used herein, the term “peripherally acting β-blocker (PABRA)” means a f adrenergic receptor antagonist or simply a β1-, β2- or non-selective β-blocker. Examples of selective peripherally acting β-blockers (PABRA) that may in certain embodiments be used in the methods disclosed herein include nadolol, atenolol, sotalol and labetalol. In certain embodiments a β-blocker that can be used in the methods herein is one or more selected from acebutolol, betaxolol, bisoprolol, celiprolol, esmolol, metaprolol and nevivolol; in other embodiments the methods do not use acebutolol, betaxolol, bisoprolol, celiprolol, esmolol, metaprolol and nevivolol as a β-blocker.

In certain embodiments a peripherally acting β-blocker (PABRA) is administered to the subject prior to the administration of a provided compound or a pharmaceutically acceptable salt thereof. In other embodiments a peripherally acting β-blocker (PABRA) is administered to the subject concurrently with the administration of a provided compound or a pharmaceutically acceptable salt thereof.

In certain embodiments of the compositions and methods provided herein, one or more peripherally acting β-blockers (PABRA) are administered prior to or concurrently with a provided compound or a pharmaceutically acceptable salt thereof in order to inhibit or preclude agonism of peripheral β1 and/or β2 adrenergic receptors by a compound of the disclosure. In some embodiments, it is preferred to block peripheral β1 and/or β2 adrenergic receptors in accordance with the compositions and methods of the present disclosure in order to preclude, or at least minimize, any adverse peripheral cardiac, metabolic or muscular effects on humans being treated.

In some embodiments of the methods provided herein, a β1 agonist and or a β2 agonist, or a non-selective β1/β2 agonist is administered to the patient in addition to a provided compound or a pharmaceutically acceptable salt thereof.

As used herein, the term “β1 agonist” is used to mean β1-adrenergic receptor agonist or β1-ADR agonist. In certain embodiments the term β1 agonist is understood to include compounds that are primarily β1 agonists, but which may also exhibit some peripheral agonism for other adrenergic receptors, such as β2-adrenergic receptors. In this application, the terms “β1-adrenergic receptor agonist”, “β1-ADR agonist”, “β1AR agonist” and “β1 agonist” may be used interchangeably. In certain embodiments, the term β1-ADR agonist expressly includes both selective and partial agonists, as well as biased and non-biased agonists. Examples of β1 adrenergic agonists include, for example, xamoterol, noradrenalin, isoprenaline, dopamine, pindolol and dobutamine and the pharmaceutically-acceptable salts of any of the above. Partial agonists and ligands of the β1-ADR are known. Further, using the methodology of Kolb et al, but for β1-ADR instead, one skilled in the art could determine new ligands by structure-based discovery. See Proc. Natl. Acad. Sci. USA 2009, 106, 6843-648.

As used herein, the term “02 agonist” is used to mean β2-adrenergic receptor agonist or β2-ADR agonist. In certain embodiments, the term β2 agonist is understood to include compounds that are primarily β2 agonists, but which may also exhibit some peripheral agonism for other adrenergic receptors, such as β1-adrenergic receptors. As used herein, the terms “β2-adrenergic receptor agonist”, “β2-ADR agonist”, “β2AR agonist” and “β2 agonist” may be used interchangeably. In some embodiments the term β2-ADR agonist expressly includes both selective and partial agonists. β2 agonists that may be used in accordance with various aspects and embodiments of the present disclosure may be short-acting, long-acting or ultra long-acting. Examples of short-acting β2 agonists that may be used are salbutamol, levosalbutamol, terbutaline, pirbuterol, procaterol, metaproterenol, bitolterol mesylate, oritodrine, isoprenaline, salmefamol, fenoterol, terbutaline, albuterol, and isoetharine. Examples of long-acting β2 agonists that may be used are salmeterol, bambuterol, formoterol and clenbuterol. Examples of ultra long-acting β2 agonists include indacaterol, vilanterol and olodaterol.

It was surprisingly found that compounds of the present disclosure exhibit unexpectedly beneficial properties, as demonstrated in the Examples section herein. For instance, it was surprisingly found that compounds of the present disclosure act as low nM (<10 nM) partial agonists of the β2 adrenergic receptor. Furthermore, compounds of the present disclosure exhibit an unexpectedly high ability to cross the blood brain barrier and accumulate in the cerebral spinal fluid. Additionally, compounds of the present disclosure exhibit excellent oral bioavailability and stability, while simultaneously exhibiting low toxicity and a low potential for drug-drug interactions.

It is believed that a provided compound or a pharmaceutically acceptable salt thereof may possess satisfactory pharmacological profile and promising biopharmaceutical properties, such as toxicological profile, metabolism and pharmacokinetic properties, solubility, and permeability. It will be understood that determination of appropriate biopharmaceutical properties is within the knowledge of a person skilled in the art, e.g., determination of cytotoxicity in cells or inhibition of certain targets or channels to determine potential toxicity.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The terms “subject”, “individual”, or “patient,” used interchangeably, refer to any animal, including mammals, preferably mice, rats, other rodents, rabbits, dogs, cats, swine, cattle, sheep, horses, or primates, and most preferably humans.

The term “therapeutically effective amount” as used herein refers to the amount of active compound or pharmaceutical agent that elicits the biological or medicinal response in a tissue, system, animal, individual or human that is being sought by a researcher, veterinarian, medical doctor or other clinician, which includes one or more of the following: (1) Preventing the disease; for example, preventing a disease, condition or disorder in an individual that may be predisposed to the disease, condition or disorder but does not yet experience or display the pathology or symptomatology of the disease, (2) Inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) Ameliorating the disease; for example, ameliorating a disease, condition or disorder in an individual that is experiencing or displaying the pathology or symptomatology of the disease, condition or disorder (i.e., reversing the pathology and/or symptomatology).

In some embodiments, the compounds of the disclosure are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

Co-Administration with One or More Other Therapeutic Agent(s)

Depending upon the particular condition, or disease, to be treated, additional therapeutic agents that are normally administered to treat that condition, can also be present in the compositions of this disclosure. As used herein, additional therapeutic agents that are normally administered to treat a particular disease, or condition, are known as “appropriate for the disease, or condition, being treated.”

In some embodiments, the present disclosure provides a method of treating a disclosed disease or condition comprising administering to a patient in need thereof an effective amount of a provided compound or a pharmaceutically acceptable salt thereof and co-administering simultaneously or sequentially an effective amount of one or more additional therapeutic agents, such as those described herein.

As used herein, the terms “combination,” “combined,” and related terms refer to the simultaneous or sequential administration of therapeutic agents in accordance with this disclosure. For example, a described compound may be administered with another therapeutic agent simultaneously or sequentially in separate unit dosage forms or together in a single unit dosage form. Accordingly, the present disclosure provides a single unit dosage form comprising a described compound, an additional therapeutic agent, and a pharmaceutically acceptable carrier, adjuvant, or vehicle. Two or more agents are typically considered to be administered “in combination” when a patient or individual is simultaneously exposed to both agents. In many embodiments, two or more agents are considered to be administered “in combination” when a patient or individual simultaneously shows therapeutically relevant levels of the agents in a particular target tissue or sample (e.g., in brain, in serum, etc.).

In some embodiments, when a provided compound or a pharmaceutically acceptable salt thereof is administered in combination therapies with other agents, they may be administered sequentially or concurrently to the patient. In some embodiments, the combination is administered sequentially. In some embodiments, the combination is administered concurrently.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the disclosure, as part of a multiple dosage regimen. Alternatively, one or more other therapeutic agents may be part of a single dosage form, mixed together with a compound of this disclosure in a single composition. If administered as a multiple dosage regime, one or more other therapeutic agent and a compound or composition of the disclosure may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the disclosure are administered as a multiple dosage regimen within greater than 24 hours apart.

In one embodiment, the present disclosure provides a composition comprising a provided compound and one or more additional therapeutic agents. The therapeutic agent may be administered together with a provided compound, or may be administered prior to or following administration of a provided compound. Suitable therapeutic agents are described in further detail below. In certain embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

The provided compounds can be administered in combination with other therapeutic agents in a variety of therapeutic applications. Therapeutic applications of interest for combination therapy include those applications in which activity of a target adrenergic receptor is the cause or a compounding factor in disease progression. As such, the subject compounds find use in combination therapies in which the inhibition of a target adrenergic receptor in the subject is desired. Examples of disease conditions which may be treated by a combination therapy including a subject compound include, but are not limited to, cardiac conditions or diseases, neurodegenerative or neurodevelopmental disease, respiratory disorders, asthma, memory impairment, depression, inflammatory diseases, stroke, ischemic brain or tissue injury and cancer. Agents of interest which can be used in jointly with the subject adrenergic receptor modulating compounds include, but are not limited to, antidepressants, antipsychotics, beta-blockers, vasoconstrictors, antihypotensives, decongestants, chemotherapeutic agents, agents used in Alzheimer's disease, and anti-inflammatory agents.

In some embodiments, a provided adrenergic receptor modulating compound can be used jointly with any agent useful in the treatment of a cardiac condition, such as cardiogenic shock, hypertension, congestive heart failure, coronary heart disease, arrhythmias, myocardial infarction or ischemic heart diseases. Agents of interest which can be used in jointly with the subject adrenergic receptor modulating compounds include, but are not limited to, denopamine, dobutamine, xamoterol, acebutolol, atenolol, betaxolol, bisoprolol, pindolol, esmolol, metoprolol, nebivolol, vortioxetine, Carvedilol, Labetalol, Phentolamine, Prazosin, Cirazoline, Methoxamine, Synephrine, Etilefrine, Metaraminol, Midodrine, and cumarin.

In some embodiments, a provided adrenergic receptor modulating compound can be used jointly with any agent useful in the treatment of a neurodegenerative or neurodevelopmental disease, such as such as Alzheimer's Disease, memory impairment, cognitive impairment, depression, stroke and ischemic brain or tissue injury, Down's syndrome or Autism. Agents of interest which can be used in jointly with a provided adrenergic receptor modulating compound include, but are not limited to, acepromazine. In some embodiments, a provided adrenergic receptor modulating compound can be used in the treatment of a disease, such as a neurodegenerative or neurodevelopmental disease, in combination with a cholinesterase inhibitor or a NMDA receptor modulators. Agents of interest include, but are not limited to, Donepezil, Aricept, Galantamine, Razadyne, Memantine, Namenda, Rivastigmine, Exelon, Tacrine and Cognex. Other agents of interest which can be used in jointly with the subject adrenergic receptor modulating compounds include, but are not limited to, 4-NEMD, 7-Me-marsanidine, Agmatine, Apraclonidine, Brimonidine, Cannabigerol, Clonidine, Detomidine, Dexmedetomidine, Fadolmidine, Guanabenz, Guanfacine, Lofexidine, Marsanidine, Medetomidine, Methamphetamine, Mivazerol, Rilmenidine, Romifidine, Talipexole, Tiamenidine, Tizanidine, Tolonidine, Xylazine, Xylometazoline, Aripiprazole, Asenapine, Atipamezole, Cirazoline, Clozapine, Efaroxan, Idazoxan, Lurasidone, Melperone, Mianserin, Mirtazapine, Napitane, Olanzapine, Paliperidone, Phenoxybenzamine, Phentolamine, Piribedil, Rauwolscine, Risperidone, Rotigotine, Quetiapine, Norquetiapine, Setiptiline, Tolazoline, Yohimbine, Ziprasidone and Zotepine. Other agents of interest which can be used in jointly with the subject adrenergic receptor modulating compounds include, but are not limited to, bitolterol, fenoterol, hexoprenaline, isoprenaline or isoproterenol, levosalbutamol or levalbuterol, orciprenaline or metaproterenol, pirbuterol, procaterol, salbutamol or albuterol, terbutaline, bambuterol, clenbuterol, formoterol, salmeterol, carmoterol, indacaterol, milveterol, olodaterol, vilanterol, fenoterol, hexoprenaline, isoxsuprine, ritodrine, salbutamol or albuterol, terbutaline, zilpaterol, ICI-118,551 and butoxamine.

The following examples are provided to further illustrate the advantages and features of the present disclosure, but they are not intended to limit the scope of the disclosure. While the examples are typical of those that might be used, other procedures, methodologies, or techniques known to those skilled in the art may alternatively be used.

### EXAMPLES

### Example 1: Synthesis of (R)-2-(((S)-3,3-dimethylbutan-2-yl)amino)-1-(1H-indazol-4-yl)ethan-1-ol

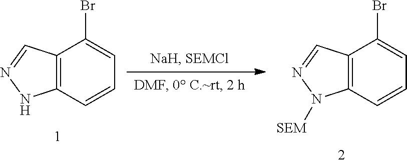 [SMILES: Brc1cccc2[nH]ncc12 ; C[Si](C)(C)CCOCn1ncc2c(Br)cccc21]

Step 1: To a solution of 1 (410 g, 2.092 mol) in DMF (4 L) was added NaH (60%, 125.5 g, 3.138 mmol) at 0° C. for 0.5 h, then SEMCl (418.5 g, 2.51 mol) was added to the above solution at 0° C., warmed to room temperature for 2 hrs. The reaction mixture was quenched with water (2.5 L) at 0° C. The mixture was heated to room temperature and stirred for 0.5 h. The mixture was extracted with dichloromethane (3*10 L) three times at rt. The combined organic phase was washed with water (2*10 L) and brine (10 L), dried over sodium sulfate, filtered, and concentrated. The residue was purified by column chromatography (1% EtOAc in PE) to give compound 2 (377 g, yield: 55.4%).

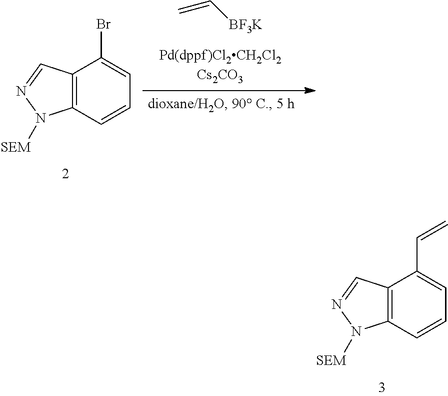

Step 2: To a stirred solution of compound 2 (475 g, 1.45 mol) in dioxane (4.7 L)/H₂O (950 ml) was added potassium trifluoro(vinyl)borate (389 g, 2.90 mol), Pd(dppf)Cl₂ (59.35 g, 72.56 mmol), Cs₂CO₃ (1416 g, 4.354 mmol) under N₂, then heated to 80° C. and stirred for 2 h. The reaction mixture was filtered, and the filtrate was extrated with EtOAc (2 L*4). The combined organic phase was washed with brine, dried over sodium sulfate, filtered, and concentrated. The residue was purified by column chromatography (5% EtOAc in PE) to give compound 3 (351 g, Yield: 88.1%).

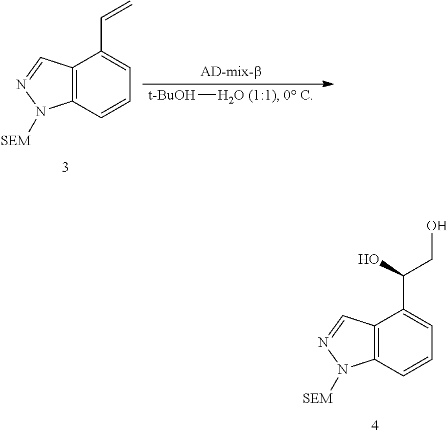

Step 3: To a solution of compound 3 (306 g, 1.115 mmol) in t-BuOH/H₂O (5508 mL/5508 mL) was added AD-mix-β (1563 g, 2.007 mol) at 0° C. Stirred for 24 h at rt. The reaction mixture was filtered, and the filtrate was washed with EtOAc (2 L*3). The combined organic phase was washed with brine, dried over sodium sulfate, filtered. The filtrate was concentrated in vacuo to afford compound 4 (350 g) as an oil and used directly in the next step.

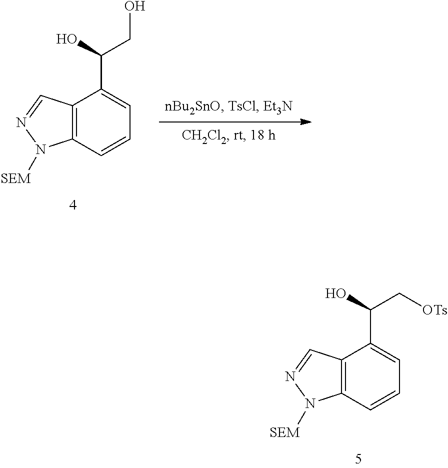 [SMILES: C[Si](C)(C)CCOCn1ncc2c([C@@H](O)CO)cccc21 ; Cc1ccc(S(=O)(=O)OC[C@H](O)c2cccc3c2cnn3COCC[Si](C)(C)C)cc1]

Step 4: To a solution of 4 (348 g, 1.128 mol) in CH₂Cl₂ (3.5 L) was added nBu₂SnO (14 g, 0.0564 mol), TsCl (258 g, 1.35 mol), Et₃N (136.7 g, 1.35 mol) at room temperature for 16 hours. The reaction solution was quenched with water (3 L). The mixture was extracted with CH₂Cl₂ (1.5 L*2). The combined organic phase was washed with brine, dried over sodium sulfate, filtered, and concentrated in vacuum. The residue was purified by column chromatography (25% EtOAc in PE) to give the compound 5 (250 g & 95 g, Yield: 57.5%).

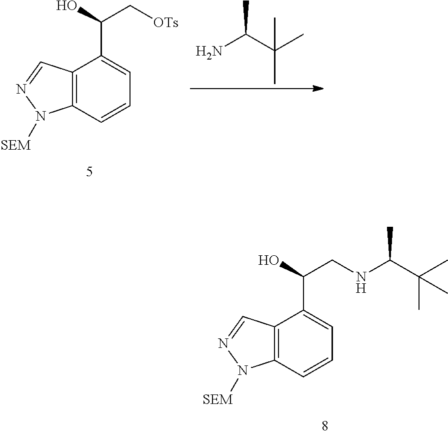

Step 5: To a solution of compound 5 (30 g, 0.065 mol) in toluene (150 mL) was added DIPEA (16.8 g, 0.13 mmol) and the reaction mixture was heated to 65° C. for 64 hrs. The reaction mixture was quenched with water (100 mL) and extracted with EtOAc (200 mL*3). The combined organic phase was washed with brine, dried over sodium sulfate, filtered and concentrated in vacuum to afford compound 8 (25 g) as an oil and used directly in the next step.

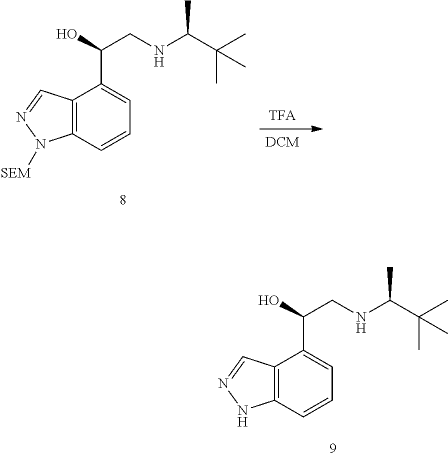 [SMILES: C[C@H](NC[C@H](O)c1cccc2c1cnn2COCC[Si](C)(C)C)C(C)(C)C ; C[C@H](NC[C@H](O)c1cccc2[nH]ncc12)C(C)(C)C]

To a solution of compound 8 (25 g) in CH₂Cl₂ (125 ml) was added TFA (125 ml) at 0-20° C. The reaction mixture was stirred at room temperature for 16 hours. The reaction mixture was concentrated in vacuum to remove CH₂Cl₂. THF (125 ml) and ammonia hydroxide (300 ml) was added in turn at 0-20° C. The reaction mixture was stirred for 12 hours. After LCMS showed the reaction mixture was completed. The reaction mixture was quenched with water (100 mL) and extracted with CH₂Cl₂ (200 mL*4). The combined organic phase was washed with brine, dried over sodium sulfate, filtered, and concentrated in vacuum. The residue was purified by column chromatography (3% MeOH in DCM) to give (R)-2-(((S)-3,3-dimethylbutan-2-yl)amino)-1-(1H-indazol-4-yl)ethan-1-ol (9, 6.8 g).

### Example 2: Evaluation of Synthesized Adrenergic Receptor Agonists

cAMP homogeneous time-resolved fluorescence (HTRF): experimental methods. Compound efficacy is determined using the cAMP Gs dynamic HTRF assay (Cisbio, catalog #62AM4PEC) largely following the manufacturer's instructions, also detailed below.

Compound preparation: Candidate beta-adrenergic compounds, dissolved to 10 mM in DMSO, are diluted in 1× stimulation buffer 1 (Cisbio Part #64SB1FDD) containing 1 mM 3-Isobutyl-1-methylxanthene (IBMX; Cayman Chemical Company, catalog #13347). Serial dilutions are made in a 96 well V-bottom polypropylene compound microplate (Corning, catalog #3363) in stimulation buffer containing 1 mM IBMX, to 2× of the final desired concentration. Standard serial dilution curves are 10-point, 5-fold dilutions starting from a highest concentration of 10 μM. Controls present on every assay plate are 0.1% DMSO (vehicle control), 1 μM isoproterenol (full beta-adrenergic agonist control) and 15 M xamoterol (partial beta-adrenergic agonist control). 5 μL from the 2× compound plate is stamped into a white 384 round well small volume HiBase assay plate (Greiner Bio-One; catalog #784075) to provide 4 technical replicates per concentration, per compound. Assay plates are centrifuged at 500×g for 10 seconds. Compounds and IBMX are prepared at 2× final dose to compensate for addition of cells.

Cell preparation: 1× stimulation buffer, washing PBS (Dulbecco's phosphate-buffered saline, − Mg − Ca; Caisson Labs, catalog #PBL01), assay PBS (Dulbecco's phosphate-buffered saline, + Mg, + Ca; Caisson Labs, catalog #PBL02) and Versene (0.02% EDTA disodium salt solution in PBS without calcium or magnesium; Caisson Labs, catalog #EDL01) are pre-warmed to 37° C. Cells expressing beta-adrenergic receptor were washed in washing PBS to remove growth medium and then released from the surface by incubating with Versene for 5-10 minutes at 37° C. Cells are harvested using assay PBS, counted manually by hemocytometer or by an automated cell counter, pelleted by centrifugation (200×g, 5 minutes) and resuspended in 37° C. 1× stimulation buffer to a final density of 1.5×10\hat{ }6 cells/mL. 5 μL of the suspended cell solution (7500 cell total) are added to all wells of the 384 well assay plate, the assay plate was covered with an Axygen® plate seal (Corning PCR-SP) and incubated in a humidified 37° C. environment supplemented with 5% CO₂ for 30 minutes.

HTRF reagent addition, reading and data analysis: After 30 minutes of cell stimulation with test compound, the assay plates are centrifuged at 500×g for 10 seconds, and incubation was stopped with the addition of 5 μL cAMP-D2 acceptor, diluted 1:21 in detection and lysis buffer 2 (Cisbio 62CL2FDF) was added to all cells. Subsequently, 5 μL Anti-cAMP-Eu Donor, diluted 1:21 in detection and lysis buffer 2, was added to cells. Plates were sealed and reactions gently ‘vortexed’ at 900 rpm on a Heidolph Titramax 1000 for at least 30 minutes at room temperature. Plates are centrifuged again at 500×g for 10 seconds, and HTRF was measured using a Tecan Spark plate reader at 50 flashes per well. HTRF ratios (665 nm/620 nm×10,000) are determined and plotted in GraphPad Prism to generate a concentration-effect curve. Potency estimates (EC₅₀ and pEC₅₀) are derived from the four-parameter nonlinear regression of the concentration-effect curve and an estimate of relative efficacy is determined by comparing the magnitude of the test compound HTRF signal window (min−max dose) with the signal window of the full agonist control, isoproterenol.

The potency and efficacy data in CHO-K1 cells are shown in Table 2 below. The potency (pEC₅₀) letter codes include: A=>8; B=>7-8; C=>6-7; D=<6. The efficacy (Emax) letter codes include: A=>90%; B=>70-90%; C=>50-70%; D=<50%.

TABLE 2
Pharmacological data of certain chemical
compounds disclosed herein.

| | | | Emax | Emax |
|---|---|---|---|---|
| | | | (% iso) | (% iso) |
| | pEC50 | pEC50 | (%) | (%) |
| I-# | [B1-AR] | [B2-AR] | [B1-AR] | [B2-AR] |
| I-1 | C | B | A | A |
| I-2 | D | B | C | A |
| I-3 | C | A | C | B |
| I-4 | D | C | D | A |
| I-5 | D | C | C | B |
| I-6 | C | A | B | A |
| I-7 | C | A | A | A |
| I-8 | D | A | B | A |
| I-9 | B | A | A | A |
| I-10 | A | A | B | A |
| I-11 | A | A | A | A |
| I-12 | A | A | A | A |
| I-13 | A | A | A | A |
| I-14 | C | A | B | C |
| I-15 | A | A | A | A |
| I-16 | B | A | A | A |
| I-17 | A | A | A | A |
| I-18 | A | A | A | A |
| I-19 | A | A | A | A |
| I-20 | A | A | A | A |
| I-21 | A | A | A | A |
| I-22 | B | A | A | A |
| I-23 | A | A | A | A |
| I-24 | B | A | A | A |
| I-25 | A | A | A | A |
| I-26 | A | A | A | A |
| I-27 | A | A | A | A |
| I-28 | A | A | A | A |
| I-29 | B | A | A | A |
| I-30 | A | A | A | A |
| I-31 | B | A | B | A |
| I-32 | B | A | A | A |
| I-33 | A | A | B | A |
| I-34 | A | A | A | B |
| I-35 | A | A | B | B |
| I-36 | A | A | B | A |
| I-37 | B | A | B | B |
| I-38 | A | A | B | A |
| I-39 | A | A | A | A |
| I-40 | B | A | B | A |
| I-41 | A | A | A | A |
| I-42 | B | A | A | B |
| I-43 | B | A | A | A |
| I-44 | A | A | A | B |
| I-45 | A | A | B | B |
| I-46 | A | A | A | A |
| I-47 | A | A | B | A |
| I-48 | A | A | A | A |
| I-49 | A | A | B | B |
| I-50 | A | A | B | B |
| I-51 | A | A | B | A |
| I-52 | C | B | B | A |
| I-53 | C | B | B | B |
| I-54 | C | B | B | B |
| I-55 | A | A | B | C |
| I-56 | B | A | C | C |
| I-57 | B | A | B | C |
| I-58 | A | A | B | B |
| I-59 | C | B | D | D |
| I-60 | A | A | B | B |
| I-61 | B | A | B | B |
| I-62 | A | A | B | B |
| I-63 | D | C | D | A |
| I-64 | C | C | B | A |
| I-65 | C | C | C | B |
| I-66 | D | C | B | A |
| I-67 | B | A | A | A |
| I-68 | B | A | B | B |
| I-69 | D | C | C | B |
| I-70 | C | B | B | C |
| I-71 | C | B | C | D |
| I-72 | C | B | C | D |
| I-73 | D | C | D | D |
| I-74 | C | D | D | D |
| I-75 | A | A | B | A |
| I-76 | A | A | B | A |
| I-77 | D | C | C | C |
| I-78 | C | C | B | A |
| I-79 | D | C | A | D |
| I-80 | C | B | B | D |
| I-81 | B | A | B | A |
| I-82 | D | D | D | D |
| I-83 | B | B | B | C |
| I-84 | B | A | B | B |
| I-85 | C | C | B | C |
| I-86 | D | C | B | B |
| I-87 | C | B | C | B |
| I-88 | C | A | B | A |
| I-89 | B | A | B | A |
| I-90 | C | B | B | A |
| I-91 | D | D | D | D |
| I-92 | B | A | B | A |
| I-93 | D | D | B | A |
| I-94 | C | C | B | B |
| I-95 | C | C | B | C |
| I-96 | A | A | B | A |
| I-97 | B | A | B | A |
| I-98 | B | A | B | B |
| I-99 | C | B | B | B |
| I-100 | A | A | B | A |
| I-101 | A | A | A | A |
| I-102 | C | A | C | D |
| I-103 | D | B | C | C |
| I-104 | D | C | B | B |
| I-105 | D | C | D | D |
| I-106 | C | B | B | C |
| I-107 | A | A | A | A |
| I-108 | B | B | A | A |
| I-109 | B | B | A | A |
| I-110 | A | A | A | A |
| I-111 | B | B | A | B |
| I-112 | A | A | A | A |
| I-113 | A | B | A | A |
| I-114 | A | A | A | A |
| I-115 | D | C | B | B |
| I-116 | B | A | B | B |
| I-117 | B | B | C | B |
| I-118 | A | A | B | B |
| I-119 | A | A | B | B |
| I-120 | A | A | B | B |
| I-121 | A | A | C | B |
| I-122 | A | A | B | B |
| I-123 | D | C | B | B |
| I-124 | D | C | B | A |
| I-125 | C | C | B | B |
| I-126 | A | A | B | A |
| I-127 | A | A | A | A |
| I-128 | A | A | A | A |
| I-129 | A | A | A | A |
| I-130 | A | A | A | A |
| I-131 | B | B | A | B |
| I-132 | A | A | A | A |
| I-133 | B | A | B | A |
| I-134 | A | A | B | A |
| I-135 | A | A | B | B |
| I-136 | A | A | A | A |
| I-137 | A | A | B | A |
| I-138 | B | A | B | A |
| I-139 | A | A | A | A |
| I-140 | A | A | A | A |
| I-141 | D | C | A | A |
| I-142 | B | B | A | A |
| I-143 | C | D | B | C |
| I-144 | D | B | B | A |
| I-145 | D | D | B | B |
| I-146 | D | D | D | A |
| I-147 | A | A | B | A |
| I-148 | D | D | B | B |
| I-149 | D | D | D | B |
| I-150 | B | A | A | A |
| I-151 | C | C | B | A |
| I-152 | B | B | B | A |
| I-153 | C | C | B | A |
| I-154 | C | A | A | A |
| I-155 | C | B | B | A |
| I-156 | C | B | B | A |
| I-157 | C | B | B | A |
| I-158 | C | C | A | A |
| I-159 | C | C | B | A |
| I-160 | C | C | B | B |
| I-161 | B | B | A | A |
| I-162 | D | C | A | A |
| I-163 | D | D | B | C |
| I-164 | C | C | B | A |
| I-165 | C | C | B | B |
| I-166 | C | B | C | B |
| I-167 | D | C | B | B |
| I-168 | B | A | B | B |
| I-169 | C | B | B | B |
| I-170 | C | B | B | B |
| I-171 | B | A | B | A |
| I-172 | C | C | B | C |
| I-173 | C | C | B | B |
| I-174 | D | C | B | C |
| I-175 | C | C | B | B |
| I-176 | D | C | C | B |
| I-177 | D | B | B | A |
| I-178 | D | C | C | A |
| I-179 | D | D | D | D |
| I-180 | D | D | D | D |
| I-181 | D | D | D | C |
| I-182 | D | D | D | C |
| I-183 | D | D | D | C |
| I-184 | D | C | B | B |
| I-185 | D | D | D | D |
| I-186 | D | D | D | D |
| I-187 | D | C | B | B |
| I-188 | C | B | A | A |
| I-189 | A | A | A | B |

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the present disclosure(s) and claims.

1. A compound according to formula I-a: 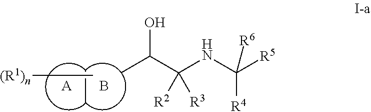
or a pharmaceutically acceptable salt thereof, wherein:
each R¹ is independently hydrogen, halogen, R^{A}, —CN, —NO₂, —SF₅, —OR, —N(R)₂, —SO₂R, —C(O)R, —C(O)N(R)₂, —NRC(O)R, —NRCO₂R, or —CO₂R;
each R is independently hydrogen or an optionally substituted group selected from C₁₋₆ aliphatic, phenyl, a 3-8 membered saturated or partially unsaturated carbocyclic or heterocyclic ring having 1-3 heteroatoms independently selected from nitrogen, oxygen, or sulfur, a 5-6 membered heteroaryl ring having 1-3 heteroatoms independently selected from nitrogen, oxygen, and sulfur, or:
two R groups on the same nitrogen atom are optionally taken together with their intervening atoms to form an optionally substituted 3-7 membered saturated or partially unsaturated heterocyclic ring having 0-2 heteroatoms, in addition to the nitrogen atom from which the two R groups are attached, independently selected from nitrogen, oxygen, and sulfur;
each R^{A} is independently an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R^{A} groups on the same carbon atom are optionally taken together with their intervening atoms to form an optionally substituted 3-6 membered saturated or partially unsaturated carbocyclic or heterocyclic ring having 1-3 heteroatoms independently selected from nitrogen, oxygen, and sulfur;
R² and R³ are each independently hydrogen or optionally substituted C₁₋₆ aliphatic, or:
R² and R³ are optionally taken together with the carbon atom they are attached to form an optionally substituted 3-6 membered saturated or partially unsaturated carbocyclic or heterocyclic ring having 1-2 heteroatoms independently selected from nitrogen, oxygen, and sulfur;
R⁴ is an optionally substituted C₁₋₆ aliphatic;
R⁵ is hydrogen or an optionally substituted C₁₋₆ aliphatic;
R⁶ is an optionally substituted group selected from C₂₋₉ aliphatic, phenylC₀₋₃alkyl, heterocyclylC₀₋₃alkyl having 1-2 heteroatoms independently selected from nitrogen, oxygen, and sulfur, and heteroarC₀₋₃alkyl having 1-2 heteroatoms independently selected from nitrogen, oxygen, and sulfur;
Ring A and Ring B, independently, are fused rings selected from benzo, 5-6 membered heteroaryl containing 1-4 heteroatoms independently selected from nitrogen, oxygen, and sulfur, and 5-7 membered saturated or partially unsaturated carbocyclyl or heterocyclyl having 1-3 heteroatoms independently selected from nitrogen, oxygen, and sulfur; and
n is 0, 1, 2, 3, 4, 5, or 6.

2. The compound of claim 1, wherein R² and R³ are each hydrogen.

3. The compound of either of claim 1, wherein R⁶ is an optionally substituted group selected from C₂₋₉ aliphatic and phenylC₀₋₃alkyl.

4. The compound of claim 1, wherein Ring A is a fused 5-6 membered heteroaryl containing 1-4 heteroatoms independently selected from nitrogen, oxygen, and sulfur.

5. The compound of claim 1, wherein Ring B is benzo or a fused 5-6 membered heteroaryl containing 1-4 heteroatoms independently selected from nitrogen, oxygen, and sulfur.

6. The compound of claim 1, wherein the compound is selected from any one of the following formulae: 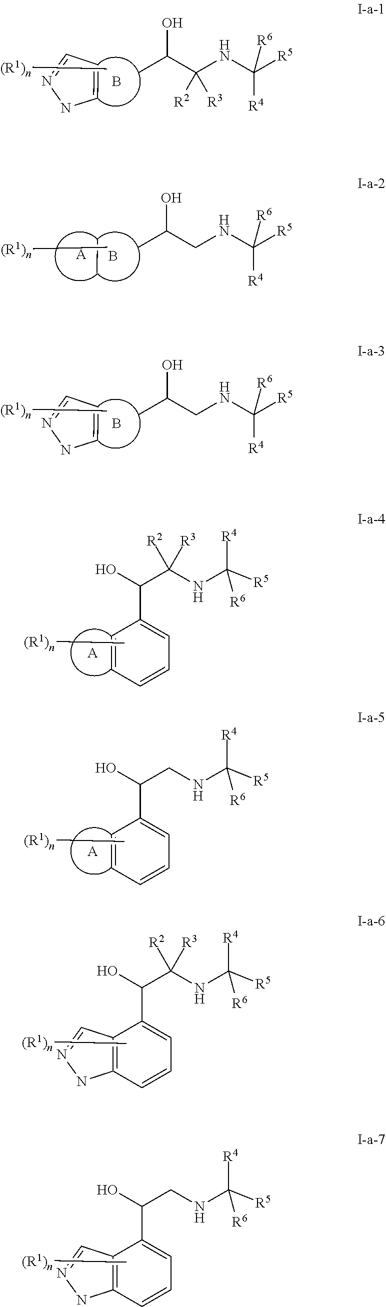
or a pharmaceutically acceptable salt thereof.

7. The compound of claim 1, wherein the compound is selected from a compound in Table 1A.

8. A compound of formula I-b: 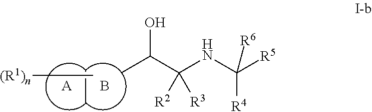
or a pharmaceutically acceptable salt thereof, wherein:
each R¹ is independently hydrogen, halogen, R^{A}, —CN, —NO₂, —SF₅, —OR, —N(R)₂, —SO₂R, —C(O)R, —C(O)N(R)₂, —NRC(O)R, —NRCO₂R, or —CO₂R;
each R is independently hydrogen or an optionally substituted group selected from C₁₋₆ aliphatic, phenyl, a 3-8 membered saturated or partially unsaturated carbocyclic or heterocyclic ring having 1-3 heteroatoms independently selected from nitrogen, oxygen, or sulfur, a 5-6 membered heteroaryl ring having 1-3 heteroatoms independently selected from nitrogen, oxygen, and sulfur, or:
two R groups on the same nitrogen atom are optionally taken together with their intervening atoms to form an optionally substituted 3-7 membered saturated or partially unsaturated heterocyclic ring having 0-2 heteroatoms, in addition to the nitrogen atom from which the two R groups are attached, independently selected from nitrogen, oxygen, and sulfur;
each R^{A} is independently an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R^{A} groups on the same carbon atom are optionally taken together with their intervening atoms to form an optionally substituted 3-6 membered saturated or partially unsaturated carbocyclic or heterocyclic ring having 1-3 heteroatoms independently selected from nitrogen, oxygen, and sulfur;
R² and R³ are each independently hydrogen or optionally substituted C₁₋₆ aliphatic, or:
R² and R³ are optionally taken together with the carbon atom they are attached to form an optionally substituted 3-6 membered saturated or partially unsaturated carbocyclic or heterocyclic ring having 1-2 heteroatoms independently selected from nitrogen, oxygen, and sulfur;
R⁴ and R⁵ are each independently hydrogen or optionally substituted C₁₋₆ aliphatic, or:
R⁴ and R⁵ are optionally taken together with the carbon atom they are attached to form an optionally substituted 3-6 membered saturated or partially unsaturated carbocyclic or heterocyclic ring having 1-2 heteroatoms independently selected from nitrogen, oxygen, and sulfur;
R⁶ is an optionally substituted group selected from C₁₋₉ aliphatic, phenylC₀₋₃alkyl, heterocyclylC₀₋₃alkyl having 1-2 heteroatoms independently selected from nitrogen, oxygen, and sulfur, and heteroarC₀₋₃alkyl having 1-2 heteroatoms independently selected from nitrogen, oxygen, and sulfur;
Ring A and Ring B, independently, are fused rings selected from benzo, 5-6 membered heteroaryl containing 1-4 heteroatoms independently selected from nitrogen, oxygen, and sulfur, and 5-7 membered saturated or partially unsaturated carbocyclyl or heterocyclyl having 1-3 heteroatoms independently selected from nitrogen, oxygen, and sulfur; and
n is 2, 3, 4, 5, or 6,
wherein when Ring A is a fused 5-membered heterocyclic ring, it is not a 5-membered heterocyclic ring containing one nitrogen.

9. The compound of claim 8, wherein R² and R³ are each hydrogen.

10. The compound of claim 8, wherein Ring A is a fused 5-6 membered heteroaryl containing 1-4 heteroatoms independently selected from nitrogen, oxygen, and sulfur.

11. The compound of claim 8, wherein Ring B is benzo or a fused 5-6 membered heteroaryl containing 1-4 heteroatoms independently selected from nitrogen, oxygen, and sulfur.

12. The compound of claim 8, wherein the compound is selected from any one of the following formulae: 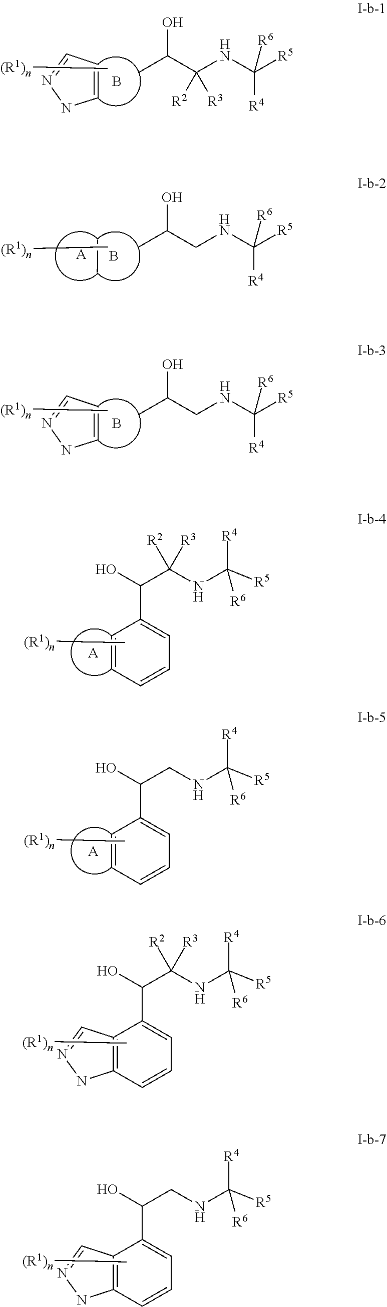
or a pharmaceutically acceptable salt thereof.

13. The compound of claim 8, wherein the compound is selected from a compound in Table 1B.

14. A compound selected from a compound in Table 1C.

15. The compound of claim 1, wherein said compound is an agonist, partial agonist or antagonist of an adrenergic receptor.

16. The compound of claim 1, wherein said compound is a β1-adrenergic receptor agonist, β2-adrenertic receptor agonist or non-selective β1/β2-adrenergic receptor agonist.

17. The compound of claim 1, wherein said compound is a β1-adrenergic receptor agonist.

18. The compound of claim 1, wherein said compound is a β2-adrenergic receptor agonist.

19. The compound of claim 1, wherein said compound is a non-selective β1/β2-adrenergic agonist.

20. A pharmaceutical composition comprising the compound of claim 1 and a pharmaceutically acceptable carrier, adjuvant, or vehicle.

21. A method of modulating an adrenergic receptor in a subject or biological sample comprising administering to said subject or contacting said biological sample with a compound of claim 1 or a pharmaceutically acceptable salt thereof, or a pharmaceutical composition thereof.

22. A method of treating an adrenergic receptor-mediated disorder, disease, and/or condition in a subject, comprising administering to the subject a therapeutically effective amount of a compound of claim 1 or a pharmaceutically acceptable salt thereof, or a pharmaceutical composition thereof.

23. The method of claim 20, wherein the adrenergic receptor-mediated disorder, disease, and/or condition is a neurodegenerative disease.

24. The method of claim 23, wherein the disease is one or more selected from MCI (mild cognitive impairment), aMCI (amnestic MCI), Vascular Dementia, Mixed Dementia, FTD (fronto-temporal dementia; Pick's disease), HD (Huntington disease), Rett Syndrome, PSP (progressive supranuclear palsy), CBD (corticobasal degeneration), SCA (spinocerebellar ataxia), MSA (Multiple system atrophy), SDS (Shy-Drager syndrome), olivopontocerebellar atrophy, TBI (traumatic brain injury), CTE (chronic traumatic encephalopathy), stroke, WKS (Wernicke-Korsakoff syndrome; alcoholic dementia & thiamine deficiency), normal pressure hydrocephalus, hypersomnia/narcolepsy, ASD (autistic spectrum disorders), FXS (fragile X syndrome), TSC (tuberous sclerosis complex), prion-related diseases (CID etc.), depressive disorders, DLB (dementia with Lewy bodies), PD (Parkinson's disease), PDD (PD dementia), ADHD (attention deficit hyperactivity disorder), Alzheimer's disease (AD), early AD, and Down Syndrome (DS).

25. The method of claim 21, wherein the subject is a human.

26. The method of claim 21, wherein the compound is administered to the subject through an oral, enteral, topical, inhalation, transmucosal, intravenous, intramuscular, intraperitoneal, subcutaneous, intranasal, epidural, intracerebral, intracerebroventricular, epicutaneous, extra-amniotic, intra-arterial, intra-articular, intracardiac, intracavernous, intradermal, intralesional, intraocular, intraosseous infusion, intraperitoneal, intrathecal, intrauterine, intravaginal, intravesical, intravitreal, transdermal, perivascular, buccal, vaginal, sublingual, or rectal route.
